(19) United States
(12) Patent Application Publication
(10) Pub. No.: US 2021/0023266 A1
(43) Pub. Date: Jan. 28, 2021

(54) TECHNIQUES FOR PROMOTING NEURONAL RECOVERY

(71) Applicant: Qrons Inc., New York, NY (US)

(72) Inventor: Ido MERFELD, Nirit (IL)

(21) Appl. No.: 17/040,953

(22) PCT Filed: Apr. 7, 2019
(86) PCT No.: PCT/IB2019/052850

§ 371 Date: Sep. 23, 2020

Related U.S. Application Data
(60) Provisional application No. 62/654,564, filed on Apr. 9, 2018.
(60) Provisional application No. 62/795,291, filed on Jan. 22, 2019.

(51) Int. Cl. A61L 27/26; A61L 27/52; A61L 27/38

(52) U.S. Cl. CPC A61L 27/26; A61L 27/52; A61L 2430/32; A61L 2300/414; A61L 2300/214; A61L 27/3834

(57) ABSTRACT

A method includes identifying a subject as having a pathological Central Nervous System (CNS) condition; and, in response, implanting, at a CNS site of the subject, a piece of a hydrogel (50). The hydrogel may be a polypseudorotaxane hydrogel. The polypseudorotaxane hydrogel may include crosslinked axles of a polymer. The polymer may include ethylene oxide (EO). The polypseudorotaxane hydrogel may further include molecules of a cyclodextrin, threaded onto the axles. Other embodiments are also described.

### CROSS-REFERENCES TO RELATED APPLICATIONS

The present application claims priority from:
U.S. Provisional Patent Application 62/654,564 to Merfeld et al., filed Apr. 9, 2018, and entitled “Techniques for promoting neuronal recovery,” and
U.S. Provisional Patent Application 62/795,291 to Merfeld et al., filed Jan. 22, 2019, and entitled “Techniques for promoting neuronal recovery.”
Both of the above applications are incorporated herein by reference.

### FIELD OF THE INVENTION

Some applications of the present invention relate in general to treatment of pathological central nervous system conditions, such as traumatic injury or neurodegenerative disease. More specifically, some applications of the present invention relate to uses of hydrogels in the treatment of such conditions.

### BACKGROUND

Traumatic injury to the central nervous system (CNS), such as traumatic brain injury (TBI) initially involves a mechanical force that causes tissue (e.g., neural tissue) damage. This can disrupt homeostasis, resulting in pathological cellular responses that can cause further damage, and/or prevent neural repair. Reactive astrogliosis, especially in more severe injuries, may inhibit neuronal growth at the injury site. For example, reactive astrocytes may form an astrocytic scar that forms a barrier around the injury site. While this barrier may beneficially serve to moderate and/or isolate the inflammatory response, it may also detrimentally inhibit influx of neural stem cells (NSC) and/or neuronal growth. (Burda et al. (2016) Experimental neurology, 275, 305-315.)

Rotaxanes and pseudorotaxanes are molecular complexes comprising a ring-shaped molecule that is threaded on an elongate molecule—in this context, often called an axle (Harrison, I. T., & Harrison, S. (1967). Journal of the American Chemical Society, 89(22), 5723-5724.). The ring-shaped molecule is inhibited from unthreading from the axle by steric effects or other molecular forces. For example, in rotaxanes, relatively bulky stoppers on the ends of the axle sterically block the ring-shaped molecule from unthreading from the axle.

Harada A. et al. ((1992) Nature 356:325) showed that rotaxanes may be formed using polymer axles comprising poly(ethylene oxide) (PEO) (also known as polyethylene glycol; PEG), with cyclodextrin (CD) serving as the ring-shaped molecule. Hydrogels based on PEO-CD compositions have been proposed for providing an injectable controlled-release matrix containing a drug (e.g., US 2002/0019369 to Li et al.). Li et al. describe that the properties of the hydrogels are tunable to affect the rate of drug release. For example, a PEO-CD hydrogel may be tailored to have a particular rate of bioabsorption. Li et al. further suggest, as example polymers for such a hydrogel, PEO block copolymers such as those with poly(propylene oxide) (PPO) including poloxamers.

### SUMMARY OF THE INVENTION

Some applications of the invention involve implanting, in a subject that has a pathological CNS condition, a bioabsorbable hydrogel, in order to promote neurogenesis and/or inhibit astrogliosis. For some applications, the condition is a traumatic injury. For some applications, the condition is a neurodegenerative disorder. The hydrogel may comprise glucose. For some applications, the hydrogel is a polypseudorotaxane hydrogel (PPRh). For some such applications, the glucose is included in the molecular structure of the hydrogel in the form of a cyclodextrin, which is threaded onto the axles of the PPRh. For some applications, each axle is a polymer comprising poly(ethylene oxide) (PEO), such as a PEO homopolymer, or copolymer such as a block copolymer, such as a poloxamer.

There are therefore provided, in accordance with various application of the present invention, the following inventive concepts:

1. A method, including:

identifying a subject as having a pathological Central Nervous System (CNS) condition; and

in response to the identifying, implanting, at a CNS site of the subject, a piece of a polypseudorotaxane hydrogel (PPRh) that includes:
crosslinked axles of a polymer including ethylene oxide (EO), and
molecules of a cyclodextrin (CD), threaded onto the axles.
   2. The method according to inventive concept 1, wherein:

the PPRh does not further include a therapeutic agent, and

implanting the piece includes implanting the piece of the PPRh that does not further include a therapeutic agent.

3. The method according to inventive concept 1, wherein:

the molecules of the CD are molecules of alpha-cyclodextrin (alpha-CD), and

implanting the piece of the PPRh includes implanting a piece of a PPRh that includes the molecules of alpha-CD, threaded onto the axles.

4. The method according to inventive concept 1, wherein:

the piece of the PPRh includes a plurality of spaces that extend from outside of the piece into the piece, and

implanting the piece includes implanting the piece that includes the plurality of spaces.

5. The method according to inventive concept 1, wherein:

the PPRh further includes one or more cell-growth nutrients; and

implanting the piece includes implanting the piece of the PPRh that further includes the one or more cell-growth nutrients.

6. The method according to inventive concept 1, wherein:

the PPRh further includes one or more neurogenesis-promoting growth factors; and

implanting the piece includes implanting the piece of the PPRh that further includes the one or more neurogenesis-promoting growth factors.

7. The method according to inventive concept 1, wherein:

the polymer is a homopolymer of EO; and

implanting the piece includes implanting the piece of the PPRh that includes the crosslinked axles of the homopolymer of EO.

8. The method according to inventive concept 1, wherein:

the piece is a macroscopic monolithic piece of the PPRh, and

implanting the piece includes implanting the macroscopic monolithic piece of the PPRh.

9. The method according to inventive concept 1, wherein:

the piece is a first microscopic piece of the PPRh, and

implanting the piece includes implanting a plurality of microscopic pieces of the PPRh, the plurality of microscopic pieces including the first microscopic piece.

10. The method according to inventive concept 1, wherein:

the piece is a first microbead of the PPRh, and

implanting the piece includes implanting a plurality of microbeads of the PPRh, the plurality of microbeads including the first microbead.

11. The method according to any one of inventive concepts 1-10, wherein:

the polymer is a copolymer that further includes propylene oxide (PO), and

implanting the piece includes implanting the piece of the PPRh that includes the copolymer that further includes PO.

12. The method according to inventive concept 11, wherein:

the copolymer is a block copolymer including (poly)ethylene oxide (PEO) and (poly)propylene oxide (PPO), and

implanting the piece includes implanting the piece of the PPRh that includes the copolymer that is the block copolymer including PEO and PPO.

13. The method according to inventive concept 12, wherein:

the block copolymer is a poloxamer, and

implanting the piece includes implanting the piece of the PPRh that includes the block copolymer that is the poloxamer.

14. The method according to any one of inventive concepts 1-13, wherein the PPRh has an elastic modulus of 0.5-10 kPa, and implanting the piece of the PPRh includes implanting the piece of the PPRh that has the elastic modulus of 0.5-10 kPa.
15. The method according to inventive concept 14, wherein the PPRh has an elastic modulus of 0.5-5 kPa, and implanting the piece of the PPRh includes implanting the piece of the PPRh that has the elastic modulus of 0.5-5 kPa.
16. The method according to inventive concept 14, wherein the PPRh has an elastic modulus of 1-10 kPa, and implanting the piece of the PPRh includes implanting the piece of the PPRh that has the elastic modulus of 1-10 kPa.
17. The method according to inventive concept 16, wherein the PPRh has an elastic modulus of 1-5 kPa, and implanting the piece of the PPRh includes implanting the piece of the PPRh that has the elastic modulus of 1-5 kPa.
18. The method according to inventive concept 17, wherein the PPRh has an elastic modulus of 1-3 kPa, and implanting the piece of the PPRh includes implanting the piece of the PPRh that has the elastic modulus of 1-3 kPa.
19. The method according to any one of inventive concepts 1-18, wherein:

the PPRh further includes L-lysine, and

implanting the piece of the PPRh includes implanting the piece of the PPRh that further includes L-lysine.

20. The method according to inventive concept 19, wherein:

the PPRh includes a solid network and a liquid component,

the L-lysine is part of the solid network, and

implanting the piece of the PPRh includes implanting the piece of the PPRh in which the L-lysine is part of the solid network.

21. The method according to inventive concept 19, wherein:

the PPRh includes a solid network and a liquid component,

the L-lysine is part of the liquid component, and

implanting the piece of the PPRh includes implanting the piece of the PPRh in which the L-lysine is part of the liquid component.

22. The method according to any one of inventive concepts 1-21, wherein:

the piece contains cells suspended in the PPRh, and

implanting the piece includes implanting the piece that contains the cells suspended in the PPRh.

23. The method according to inventive concept 22, wherein:

the cells are mesenchymal stem cells (MSC), and

implanting the piece includes implanting the piece that contains the MSC suspended in the PPRh.

24. The method according to any one of inventive concepts 1-23, wherein:

the condition includes astrogliosis, and

identifying the subject includes identifying the subject as having the condition that includes astrogliosis.

25. The method according to any one of inventive concepts 1-23, wherein:

the condition includes neuronal loss, and

identifying the subject includes identifying the subject as having the condition that includes neuronal loss.

26. The method according to any one of inventive concepts 1-23, wherein:

the condition is a glioma, and

identifying the subject includes identifying the subject as having the glioma.

27. The method according to any one of inventive concepts 1-23, wherein:

the condition is a stroke, and

identifying the subject as having the condition includes identifying the subject as having had the stroke.

28. The method according to any one of inventive concepts 1-23, wherein:

the condition is a neurodegenerative disorder, and

identifying the subject as having the condition includes identifying the subject as having the neurodegenerative disorder.

29. The method according to inventive concept 28, wherein the neurodegenerative disorder is a neurodegenerative disorder resulting from a traumatic brain injury (TBI), and

identifying the subject as having the condition includes identifying the subject as having the neurodegenerative disorder resulting from the traumatic brain injury (TBI).

30. The method according to inventive concept 28, wherein:

the neurodegenerative disorder is a neurodegenerative disorder that involves astrogliosis, and

identifying the subject as having the neurodegenerative disorder includes identifying the subject as having the neurodegenerative disorder that involves astrogliosis.

31. The method according to inventive concept 28, wherein:

the neurodegenerative disorder is amyotrophic lateral sclerosis, and

identifying the subject as having the neurodegenerative disorder includes identifying the subject as having amyotrophic lateral sclerosis.

32. The method according to inventive concept 28, wherein:

the neurodegenerative disorder is Parkinson's disease, and

identifying the subject as having the neurodegenerative disorder includes identifying the subject as having Parkinson's disease.

33. The method according to inventive concept 28, wherein:

the neurodegenerative disorder is Alzheimer's disease, and

identifying the subject as having the neurodegenerative disorder includes identifying the subject as having Parkinson's disease.

34. The method according to inventive concept 28, wherein:

the neurodegenerative disorder is Huntington's disease, and

identifying the subject as having the neurodegenerative disorder includes identifying the subject as having Huntington's disease.

35. The method according to any one of inventive concepts 1-23, wherein:

the condition is a traumatic CNS injury, and

identifying the subject as having the condition includes identifying the subject as having the traumatic CNS injury.

36. The method according to inventive concept 35, wherein:

the traumatic CNS injury is a traumatic brain injury (TBI), and

identifying the subject as having the traumatic CNS injury includes identifying the subject as having the TBI.

37. The method according to inventive concept 35, wherein:

the traumatic CNS injury is a focal injury at an injury site, and

implanting the piece includes implanting the piece at the injury site.

38. The method according to inventive concept 35, wherein:

the traumatic CNS injury is a penetrating injury resulting in a cavity, and

implanting the piece includes implanting the piece in the cavity.

39. The method according to inventive concept 35, wherein:

the traumatic CNS injury is a diffuse brain injury, and

implanting the piece includes implanting the piece between a skull and a brain of the subject.

40. The method according to inventive concept 35, further including identifying when a traumatic incident that caused the traumatic CNS injury occurred, and wherein implanting the piece includes implanting the piece 0-4 days after the traumatic incident occurred.
41. The method according to any one of inventive concepts 1-23, wherein implanting the piece includes implanting the piece in contact with grey matter of the CNS of the subject.
42. The method according to any one of inventive concepts 1-23, wherein implanting the piece includes implanting the piece in contact with white matter of the CNS of the subject.
43. The method according to any one of inventive concepts 1-23, wherein implanting the piece includes implanting the piece at a brain of the subject.
44. The method according to inventive concept 43, wherein implanting the piece includes implanting the piece within a sulcus of the brain of the subject.
45. The method according to inventive concept 43, wherein implanting the piece includes implanting the piece within a longitudinal fissure of the brain of the subject.
46. The method according to inventive concept 43, wherein implanting the piece includes implanting the piece at a corpus callosum of the brain of the subject.
47. The method according to inventive concept 43, wherein implanting the piece includes implanting the piece at a cerebrum of the brain of the subject.
48. The method according to inventive concept 47, wherein implanting the piece includes implanting the piece at a cerebral cortex of the brain of the subject.
49. The method according to inventive concept 47, wherein implanting the piece includes implanting the piece at a hippocampus of the brain of the subject.
50. The method according to inventive concept 47, wherein implanting the piece includes implanting the piece at a basal ganglia of the brain of the subject.
51. The method according to inventive concept 47, wherein implanting the piece includes implanting the piece at an olfactory bulb of the brain of the subject.
52. The method according to inventive concept 47, wherein implanting the piece includes implanting the piece at a frontal lobe of the brain of the subject.
53. The method according to inventive concept 47, wherein implanting the piece includes implanting the piece at a parietal lobe of the brain of the subject.
54. The method according to inventive concept 47, wherein implanting the piece includes implanting the piece at a temporal lobe of the brain of the subject.
55. The method according to inventive concept 47, wherein implanting the piece includes implanting the piece at an occipital lobe of the brain of the subject.
56. The method according to inventive concept 43, wherein implanting the piece includes implanting the piece at a diencephalon of the brain of the subject.
57. The method according to inventive concept 43, wherein implanting the piece includes implanting the piece at a mesencephalon of the brain of the subject.
58. The method according to inventive concept 43, wherein implanting the piece includes implanting the piece at a cerebellum of the brain of the subject.
59. The method according to inventive concept 43, wherein implanting the piece includes implanting the piece at a pons of the brain of the subject.
60. The method according to inventive concept 43, wherein implanting the piece includes implanting the piece at a medulla oblongata of the brain of the subject.
61. The method according to any one of inventive concepts 1-23, wherein implanting the piece includes implanting the piece in a spinal cord of the subject.
62. The method according to inventive concept 61, wherein implanting the piece includes implanting the piece in an epidural space of the spinal cord of the subject.
63. The method according to any one of inventive concepts 1-23, wherein:

the piece is a patch, and

implanting the piece includes implanting the patch between a skull and a brain of the subject.

64. The method according to inventive concept 63, wherein implanting the patch includes implanting the patch in a subarachnoid space of the subject.
65. The method according to inventive concept 63, wherein implanting the patch includes implanting the patch in a subdural space of the subject.
66. The method according to any one of inventive concepts 1-65, further including identifying the CNS site in response to imaging data.
67. The method according to inventive concept 66, further including generating the imaging data using an imaging technique.
68. The method according to inventive concept 66, further including shaping the piece in response to the imaging data.
69. The method according to inventive concept 68, wherein shaping the piece includes sculpting the piece.
70. The method according to inventive concept 68, wherein shaping the piece includes 3D-printing the piece.
71. A method, including:

identifying a subject as having a pathological Central Nervous System (CNS) condition; and

in response to the identifying, implanting, at a CNS site of the subject, a piece of a hydrogel that includes glucose.

72. The method according to inventive concept 71, wherein:

the condition includes astrogliosis, and

identifying the subject includes identifying the subject as having the condition that includes astrogliosis.

73. The method according to inventive concept 71, wherein:

the condition includes neuronal loss, and

identifying the subject includes identifying the subject as having the condition that includes neuronal loss.

74. The method according to inventive concept 71, wherein:

the condition is a glioma, and

identifying the subject includes identifying the subject as having the glioma.

75. The method according to inventive concept 71, wherein:

the condition is a stroke, and

identifying the subject as having the condition includes identifying the subject as having had the stroke.

76. The method according to inventive concept 71, wherein:

the condition is a neurodegenerative disorder, and

identifying the subject as having the condition includes identifying the subject as having the neurodegenerative disorder.

77. The method according to inventive concept 71, wherein:

the condition is amyotrophic lateral sclerosis, and

identifying the subject as having the condition includes identifying the subject as having amyotrophic lateral sclerosis.

78. The method according to inventive concept 71, wherein:

the condition is Parkinson's disease, and

identifying the subject as having the condition includes identifying the subject as having Parkinson's disease.

79. The method according to inventive concept 71, wherein:

the condition is Alzheimer's disease, and

identifying the subject as having the condition includes identifying the subject as having Alzheimer's disease.

80. The method according to inventive concept 71, wherein:

the condition is Huntington's disease, and

identifying the subject as having the condition includes identifying the subject as having Huntington's disease.

81. The method according to any one of inventive concepts 71-80, wherein:

the piece is a macroscopic monolithic piece of the hydrogel, and

implanting the piece includes implanting the macroscopic monolithic piece of the hydrogel.

82. The method according to any one of inventive concepts 71-80, wherein:

the piece is a first microscopic piece of the hydrogel, and

implanting the piece includes implanting a plurality of microscopic pieces of the hydrogel, the plurality of microscopic pieces including the first microscopic piece.

83. The method according to any one of inventive concepts 71-80, wherein:

the piece is a first microbead of the hydrogel, and

implanting the piece includes implanting a plurality of microbeads of the hydrogel, the plurality of microbeads including the first microbead.

84. The method according to any one of inventive concepts 71-83, wherein:

the hydrogel further includes one or more cell-growth nutrients; and

implanting the piece includes implanting the piece of the hydrogel that further includes the one or more cell-growth nutrients.

85. The method according to any one of inventive concepts 71-84, wherein:

the hydrogel further includes one or more neurogenesis-promoting growth factors;

and implanting the piece includes implanting the piece of the hydrogel that further includes the one or more neurogenesis-promoting growth factors.

86. The method according to any one of inventive concepts 71-83, wherein:

the hydrogel does not further include a therapeutic agent, and

implanting the piece includes implanting the piece of the hydrogel that does not further include a therapeutic agent.

87. The method according to any one of inventive concepts 71-86, wherein:

the glucose is in the form of a cyclodextrin (CD); and

implanting the hydrogel includes implanting the hydrogel in which the glucose is in the form of the CD.

88. The method according to any one of inventive concepts 71-87, wherein:

the piece of the hydrogel includes a plurality of spaces that extend from outside of the piece into the piece, and

implanting the piece includes implanting the piece that includes the plurality of spaces.

89. The method according to any one of inventive concepts 71-88, wherein implanting the piece includes implanting the piece in contact with grey matter of the CNS of the subject.
90. The method according to any one of inventive concepts 71-88, wherein implanting the piece includes implanting the piece in contact with white matter of the CNS of the subject.
91. The method according to any one of inventive concepts 71-90, wherein the hydrogel has an elastic modulus of 0.5-10 kPa, and implanting the piece of the hydrogel includes implanting the piece of the hydrogel that has the elastic modulus of 0.5-10 kPa.
92. The method according to inventive concept 91, wherein the hydrogel has an elastic modulus of 0.5-5 kPa, and implanting the piece of the hydrogel includes implanting the piece of the hydrogel that has the elastic modulus of 0.5-5 kPa.
93. The method according to inventive concept 91, wherein the hydrogel has an elastic modulus of 1-10 kPa, and implanting the piece of the hydrogel includes implanting the piece of the hydrogel that has the elastic modulus of 1-10 kPa.
94. The method according to inventive concept 93, wherein the hydrogel has an elastic modulus of 1-5 kPa, and implanting the piece of the hydrogel includes implanting the piece of the hydrogel that has the elastic modulus of 1-5 kPa.
95. The method according to inventive concept 94, wherein the hydrogel has an elastic modulus of 1-3 kPa, and implanting the piece of the hydrogel includes implanting the piece of the hydrogel that has the elastic modulus of 1-3 kPa.
96. The method according to any one of inventive concepts 71-95, wherein:

the hydrogel further includes L-lysine, and

implanting the piece of the hydrogel includes implanting a piece of the hydrogel that further includes L-lysine.

97. The method according to inventive concept 96, wherein:

the hydrogel includes a solid network and a liquid component, and the L-lysine is part of the solid network; and

implanting the piece of the hydrogel includes implanting a piece of the hydrogel in which the L-lysine is part of the solid network.

98. The method according to inventive concept 96, wherein:

the hydrogel includes a solid network and a liquid component, and the L-lysine is part of the liquid component; and

implanting the piece of the hydrogel includes implanting a piece of the hydrogel in which the L-lysine is part of the liquid component.

99. The method according to any one of inventive concepts 71-95, wherein:

the hydrogel includes a solid network and a liquid component, and the glucose is part of the solid network; and

implanting the hydrogel includes implanting the hydrogel in which the glucose is part of the solid network.

100. The method according to inventive concept 99, wherein:

the hydrogel is a polypseudorotaxane hydrogel (PPRh) that includes crosslinked axles, and molecules of a cyclodextrin (CD) threaded onto the axles, and

implanting the hydrogel includes implanting the PPRh that includes the crosslinked axles, and the molecules of the cyclodextrin (CD) threaded onto the axles.

101. The method according to inventive concept 100, wherein:

the CD is alpha-CD; and

implanting the PPRh includes implanting the PPRh that includes the molecules of alpha-CD threaded onto the axles.

102. The method according to inventive concept 100, wherein:

each of the axles is an axle of a polymer including ethylene oxide (EO), and

implanting the PPRh includes implanting the PPRh that includes the axles of the polymer including EO.

103. The method according to any one of inventive concepts 71-102, wherein:

the condition is a traumatic CNS injury, and

identifying the subject as having the condition includes identifying the subject as having the traumatic CNS injury.

104. The method according to inventive concept 103, wherein:

the traumatic CNS injury is a traumatic brain injury (TBI), and

identifying the subject as having the traumatic CNS injury includes identifying the subject as having the TBI.

105. The method according to inventive concept 104, wherein:

the TBI is a diffuse brain injury, and

implanting the piece includes implanting the piece between a skull and a brain of the subject.

106. The method according to inventive concept 103, wherein:

the traumatic CNS injury is a focal injury at an injury site, and

implanting the piece includes implanting the piece at the injury site.

107. The method according to inventive concept 103, wherein:

the traumatic CNS injury is a penetrating injury resulting in a cavity, and

implanting the piece includes implanting the piece in the cavity.

108. The method according to inventive concept 103, further including identifying when a traumatic incident that caused the traumatic CNS injury occurred, and wherein implanting the piece includes implanting the piece 0-4 days after the traumatic incident occurred.
109. The method according to any one of inventive concepts 71-102, wherein implanting the piece includes implanting the piece at a brain of the subject.
110. The method according to inventive concept 109, wherein implanting the piece includes implanting the piece within a sulcus of the brain of the subject.
111. The method according to inventive concept 109, wherein implanting the piece includes implanting the piece within a longitudinal fissure of the brain of the subject.
112. The method according to inventive concept 109, wherein implanting the piece includes implanting the piece at a corpus callosum of the brain of the subject.
113. The method according to inventive concept 109, wherein implanting the piece includes implanting the piece at a cerebrum of the brain of the subject.
114. The method according to inventive concept 113, wherein implanting the piece includes implanting the piece at a cerebral cortex of the brain of the subject.
115. The method according to inventive concept 113, wherein implanting the piece includes implanting the piece at a hippocampus of the brain of the subject.
116. The method according to inventive concept 113, wherein implanting the piece includes implanting the piece at a basal ganglia of the brain of the subject.
117. The method according to inventive concept 113, wherein implanting the piece includes implanting the piece at an olfactory bulb of the brain of the subject.
118. The method according to inventive concept 113, wherein implanting the piece includes implanting the piece at a frontal lobe of the brain of the subject.
119. The method according to inventive concept 113, wherein implanting the piece includes implanting the piece at a parietal lobe of the brain of the subject.
120. The method according to inventive concept 113, wherein implanting the piece includes implanting the piece at a temporal lobe of the brain of the subject.
121. The method according to inventive concept 113, wherein implanting the piece includes implanting the piece at an occipital lobe of the brain of the subject.
122. The method according to inventive concept 109, wherein implanting the piece includes implanting the piece at a diencephalon of the brain of the subject.
123. The method according to inventive concept 109, wherein implanting the piece includes implanting the piece at a mesencephalon of the brain of the subject.
124. The method according to inventive concept 109, wherein implanting the piece includes implanting the piece at a cerebellum of the brain of the subject.
125. The method according to inventive concept 109, wherein implanting the piece includes implanting the piece at a pons of the brain of the subject.
126. The method according to inventive concept 109, wherein implanting the piece includes implanting the piece at a medulla oblongata of the brain of the subject.
127. The method according to any one of inventive concepts 71-102, wherein implanting the piece includes implanting the piece in a spinal cord of the subject.
128. The method according to inventive concept 127, wherein implanting the piece includes implanting the piece in an epidural space of the spinal cord of the subject.
129. The method according to any one of inventive concepts 71-102, wherein:

the piece contains cells suspended in the hydrogel, and

implanting the piece includes implanting the piece that contains the cells suspended in the hydrogel.

130. The method according to inventive concept 129, wherein:

the cells are mesenchymal stem cells (MSC), and

implanting the piece includes implanting the piece that contains the MSC suspended in the hydrogel.

131. The method according to any one of inventive concepts 71-102, further including identifying the CNS site in response to imaging data.
132. The method according to inventive concept 131, further including generating the imaging data using an imaging technique.
133. The method according to inventive concept 131, further including shaping the piece in response to the imaging data.
134. The method according to inventive concept 133, wherein shaping the piece includes sculpting the piece.
135. The method according to inventive concept 133, wherein shaping the piece includes 3D-printing the piece.
136. The method according to any one of inventive concepts 71-102, wherein:

the piece is a patch, and

implanting the piece includes implanting the patch between a skull and a brain of the subject.

137. The method according to inventive concept 136, wherein implanting the patch includes implanting the patch in a subarachnoid space of the subject.
138. The method according to inventive concept 136, wherein implanting the patch includes implanting the patch in a subdural space of the subject.
139. A method, including:

identifying a subject as having reactive astrogliosis; and

in response to the identifying, implanting, at a CNS site of the subject, a piece of a hydrogel that includes glucose.

140. The method according to inventive concept 139, wherein:

the piece is a macroscopic monolithic piece of the hydrogel, and

implanting the piece includes implanting the macroscopic monolithic piece of the hydrogel.

141. The method according to inventive concept 139, wherein:

the piece is a first microscopic piece of the hydrogel, and

implanting the piece includes implanting a plurality of microscopic pieces of the hydrogel, the plurality of microscopic pieces including the first microscopic piece.

142. The method according to inventive concept 139, wherein:

the piece is a first microbead of the hydrogel, and

implanting the piece includes implanting a plurality of microbeads of the hydrogel, the plurality of microbeads including the first microbead.

143. The method according to inventive concept 139, wherein:

the hydrogel further includes one or more cell-growth nutrients; and

implanting the piece includes implanting the piece of the hydrogel that further includes the one or more cell-growth nutrients.

144. The method according to inventive concept 139, wherein:

the hydrogel further includes one or more neurogenesis-promoting growth factors;

and implanting the piece includes implanting the piece of the hydrogel that further includes the one or more neurogenesis-promoting growth factors.

145. The method according to inventive concept 139, wherein:

the hydrogel does not further include a therapeutic agent, and

implanting the piece includes implanting the piece of the hydrogel that does not further include a therapeutic agent.

146. The method according to inventive concept 139, wherein:

the glucose is in the form of a cyclodextrin (CD); and

implanting the hydrogel includes implanting the hydrogel in which the glucose is in the form of the CD.

147. The method according to inventive concept 139, wherein:

the piece of the hydrogel includes a plurality of spaces that extend from outside of the piece into the piece, and

implanting the piece includes implanting the piece that includes the plurality of spaces.

148. The method according to inventive concept 139, wherein the hydrogel has an elastic modulus of 0.5-10 kPa, and implanting the piece of the hydrogel includes implanting the piece of the hydrogel that has the elastic modulus of 0.5-10 kPa.
149. The method according to inventive concept 148, wherein the hydrogel has an elastic modulus of 0.5-5 kPa, and implanting the piece of the hydrogel includes implanting the piece of the hydrogel that has the elastic modulus of 0.5-5 kPa.
150. The method according to inventive concept 148, wherein the hydrogel has an elastic modulus of 1-10 kPa, and implanting the piece of the hydrogel includes implanting the piece of the hydrogel that has the elastic modulus of 1-10 kPa.
151. The method according to inventive concept 150, wherein the hydrogel has an elastic modulus of 1-5 kPa, and implanting the piece of the hydrogel includes implanting the piece of the hydrogel that has the elastic modulus of 1-5 kPa.
152. The method according to inventive concept 151, wherein the hydrogel has an elastic modulus of 1-3 kPa, and implanting the piece of the hydrogel includes implanting the piece of the hydrogel that has the elastic modulus of 1-3 kPa.
153. The method according to any one of inventive concepts 139-152, wherein:

the hydrogel further includes L-lysine, and

implanting the piece of the hydrogel includes implanting a piece of the hydrogel that further includes L-lysine.

154. The method according to inventive concept 153, wherein:

the hydrogel includes a solid network and a liquid component, and the L-lysine is part of the solid network; and

implanting the piece of the hydrogel includes implanting a piece of the hydrogel in which the L-lysine is part of the solid network.

155. The method according to inventive concept 153, wherein:

the hydrogel includes a solid network and a liquid component, and the L-lysine is part of the liquid component; and

implanting the piece of the hydrogel includes implanting a piece of the hydrogel in which the L-lysine is part of the liquid component.

156. The method according to any one of inventive concepts 139-155, wherein:

the hydrogel includes a solid network and a liquid component, and the glucose is part of the solid network; and

implanting the hydrogel includes implanting the hydrogel in which the glucose is part of the solid network.

157. The method according to inventive concept 156, wherein:

the hydrogel is a polypseudorotaxane hydrogel (PPRh) that includes crosslinked axles, and molecules of a cyclodextrin (CD) threaded onto the axles, and

implanting the hydrogel includes implanting the PPRh that includes the crosslinked axles, and the molecules of the cyclodextrin (CD) threaded onto the axles.

158. The method according to inventive concept 157, wherein:

the CD is alpha-CD; and

implanting the PPRh includes implanting the PPRh that includes the molecules of alpha-CD threaded onto the axles.

159. The method according to inventive concept 157, wherein:

each of the axles is an axle of a polymer including ethylene oxide (EO), and

implanting the PPRh includes implanting the PPRh that includes the axles of the polymer including EO.

160. The method according to any one of inventive concepts 139-159, wherein:

the piece contains cells suspended in the hydrogel, and

implanting the piece includes implanting the piece that contains the cells suspended in the hydrogel.

161. The method according to inventive concept 160, wherein:

the cells are mesenchymal stem cells (MSC), and

implanting the piece includes implanting the piece that contains the MSC suspended in the hydrogel.

162. The method according to any one of inventive concepts 139-161, further including identifying the CNS site in response to imaging data.
163. The method according to inventive concept 162, further including generating the imaging data using an imaging technique.
164. The method according to any one of inventive concepts 139-163, further including shaping the piece in response to imaging data.
165. The method according to inventive concept 164, wherein shaping the piece includes sculpting the piece.
166. The method according to inventive concept 164, wherein shaping the piece includes 3D-printing the piece.
167. A method, including:

locating an astrocytic scar within a Central Nervous System (CNS) of a subject; and

in response to the locating, implanting, adjacent to the scar, a piece of a hydrogel that includes glucose.

168. The method according to inventive concept 167, wherein:

the piece is a macroscopic monolithic piece of the hydrogel, and

implanting the piece includes implanting the macroscopic monolithic piece of the hydrogel.

169. The method according to inventive concept 167, wherein:

the piece is a first microscopic piece of the hydrogel, and

implanting the piece includes implanting a plurality of microscopic pieces of the hydrogel, the plurality of microscopic pieces including the first microscopic piece.

170. The method according to inventive concept 167, wherein:

the piece is a first microbead of the hydrogel, and

implanting the piece includes implanting a plurality of microbeads of the hydrogel, the plurality of microbeads including the first microbead.

171. The method according to inventive concept 167, wherein:

the hydrogel further includes one or more cell-growth nutrients; and

implanting the piece includes implanting the piece of the hydrogel that further includes the one or more cell-growth nutrients.

172. The method according to inventive concept 167, wherein:

the hydrogel further includes one or more neurogenesis-promoting growth factors; and

implanting the piece includes implanting the piece of the hydrogel that further includes the one or more neurogenesis-promoting growth factors.

173. The method according to inventive concept 167, wherein:

the hydrogel does not further include a therapeutic agent, and

implanting the piece includes implanting the piece of the hydrogel that does not further include a therapeutic agent.

174. The method according to inventive concept 167, wherein:

the glucose is in the form of a cyclodextrin (CD); and

implanting the hydrogel includes implanting the hydrogel in which the glucose is in the form of the CD.

175. The method according to inventive concept 167, wherein:

the piece of the hydrogel includes a plurality of spaces that extend from outside of the piece into the piece, and

implanting the piece includes implanting the piece that includes the plurality of spaces.

176. The method according to any one of inventive concepts 167-175, wherein the hydrogel has an elastic modulus of 0.5-10 kPa, and implanting the piece of the hydrogel includes implanting the piece of the hydrogel that has the elastic modulus of 0.5-10 kPa.
177. The method according to inventive concept 176, wherein the hydrogel has an elastic modulus of 0.5-5 kPa, and implanting the piece of the hydrogel includes implanting the piece of the hydrogel that has the elastic modulus of 0.5-5 kPa.
178. The method according to inventive concept 176, wherein the hydrogel has an elastic modulus of 1-10 kPa, and implanting the piece of the hydrogel includes implanting the piece of the hydrogel that has the elastic modulus of 1-10 kPa.
179. The method according to inventive concept 178, wherein the hydrogel has an elastic modulus of 1-5 kPa, and implanting the piece of the hydrogel includes implanting the piece of the hydrogel that has the elastic modulus of 1-5 kPa.
180. The method according to inventive concept 179, wherein the hydrogel has an elastic modulus of 1-3 kPa, and implanting the piece of the hydrogel includes implanting the piece of the hydrogel that has the elastic modulus of 1-3 kPa.
181. The method according to any one of inventive concepts 167-180, wherein:

the hydrogel further includes L-lysine, and

implanting the piece of the hydrogel includes implanting a piece of the hydrogel that further includes L-lysine.

182. The method according to inventive concept 181, wherein:

the hydrogel includes a solid network and a liquid component, and the L-lysine is part of the solid network; and

implanting the piece of the hydrogel includes implanting a piece of the hydrogel in which the L-lysine is part of the solid network.

183. The method according to inventive concept 181, wherein:

the hydrogel includes a solid network and a liquid component, and the L-lysine is part of the liquid component; and

implanting the piece of the hydrogel includes implanting a piece of the hydrogel in which the L-lysine is part of the liquid component.

184. The method according to any one of inventive concepts 167-183, wherein implanting the piece of the hydrogel adjacent to the scar includes implanting the piece of the hydrogel within 1 mm of the scar.
185. The method according to inventive concept 184, wherein implanting the piece of the hydrogel adjacent to the scar includes implanting the piece of the hydrogel within 100 microns of the scar.
186. The method according to any one of inventive concepts 167-185, wherein:

the hydrogel includes a solid network and a liquid component, and the glucose is part of the solid network; and

implanting the hydrogel includes implanting the hydrogel in which the glucose is part of the solid network.

187. The method according to inventive concept 186, wherein:

the hydrogel is a polypseudorotaxane hydrogel (PPRh) that includes crosslinked axles, and molecules of a cyclodextrin (CD) threaded onto the axles, and

implanting the hydrogel includes implanting the PPRh that includes the crosslinked axles, and the molecules of the cyclodextrin (CD) threaded onto the axles.

188. The method according to inventive concept 187, wherein:

the CD is alpha-CD; and

implanting the PPRh includes implanting the PPRh that includes the molecules of alpha-CD threaded onto the axles.

189. The method according to inventive concept 187, wherein:

each of the axles is an axle of a polymer including ethylene oxide (EO), and

implanting the PPRh includes implanting the PPRh that includes the axles of the polymer including EO.

190. The method according to any one of inventive concepts 167-189, wherein:

the piece contains cells suspended in the hydrogel, and

implanting the piece includes implanting the piece that contains the cells suspended in the hydrogel.

191. The method according to inventive concept 190, wherein:

the cells are mesenchymal stem cells (MSC), and

implanting the piece includes implanting the piece that contains the MSC suspended in the hydrogel.

192. The method according to any one of inventive concepts 167-191, further including identifying the CNS site in response to imaging data.
193. The method according to inventive concept 192, further including generating the imaging data using an imaging technique.
194. The method according to inventive concept 192, further including shaping the piece in response to the imaging data.
195. The method according to inventive concept 194, wherein shaping the piece includes sculpting the piece.
196. The method according to inventive concept 194, wherein shaping the piece includes 3D-printing the piece.
197. A method, including:

identifying a subject as having a glioma at a CNS site of the subject; and

in response to the identifying, implanting, at the CNS site, a piece of a hydrogel that includes glucose.

198. The method according to inventive concept 197, wherein the hydrogel has an elastic modulus of 0.5-10 kPa, and implanting the piece of the hydrogel includes implanting the piece of the hydrogel that has the elastic modulus of 0.5-10 kPa.
199. The method according to inventive concept 198, wherein the hydrogel has an elastic modulus of 0.5-5 kPa, and implanting the piece of the hydrogel includes implanting the piece of the hydrogel that has the elastic modulus of 0.5-5 kPa.
200. The method according to inventive concept 198, wherein the hydrogel has an elastic modulus of 1-10 kPa, and implanting the piece of the hydrogel includes implanting the piece of the hydrogel that has the elastic modulus of 1-10 kPa.
201. The method according to inventive concept 200, wherein the hydrogel has an elastic modulus of 1-5 kPa, and implanting the piece of the hydrogel includes implanting the piece of the hydrogel that has the elastic modulus of 1-5 kPa.
202. The method according to inventive concept 201, wherein the hydrogel has an elastic modulus of 1-3 kPa, and implanting the piece of the hydrogel includes implanting the piece of the hydrogel that has the elastic modulus of 1-3 kPa.
203. The method according to any one of inventive concepts 197-202, wherein:

the hydrogel further includes L-lysine, and

implanting the piece of the hydrogel includes implanting a piece of the hydrogel that further includes L-lysine.

204. The method according to inventive concept 203, wherein:

the hydrogel includes a solid network and a liquid component, and the L-lysine is part of the solid network; and

implanting the piece of the hydrogel includes implanting a piece of the hydrogel in which the L-lysine is part of the solid network.

205. The method according to inventive concept 203, wherein:

the hydrogel includes a solid network and a liquid component, and the L-lysine is part of the liquid component; and

implanting the piece of the hydrogel includes implanting a piece of the hydrogel in which the L-lysine is part of the liquid component.

206. The method according to any one of inventive concepts 197-205, wherein:

the glioma is an astrocytoma, and

identifying the subject as having the glioma includes identifying the subject as having the astrocytoma.

207. The method according to any one of inventive concepts 197-206, further including treating the glioma prior to implanting the piece of the hydrogel.
208. The method according to inventive concept 207, wherein treating the glioma includes removing at least part of the glioma.
209. The method according to inventive concept 207, wherein treating the glioma includes ablating at least part of the glioma.
210. The method according to any one of inventive concepts 197-209, wherein:

the piece is a macroscopic monolithic piece of the hydrogel, and

implanting the piece includes implanting the macroscopic monolithic piece of the hydrogel.

211. The method according to any one of inventive concepts 197-210, wherein:

the piece is a first microscopic piece of the hydrogel, and

implanting the piece includes implanting a plurality of microscopic pieces of the hydrogel, the plurality of microscopic pieces including the first microscopic piece.

212. The method according to any one of inventive concepts 197-211, wherein:

the piece is a first microbead of the hydrogel, and

implanting the piece includes implanting a plurality of microbeads of the hydrogel, the plurality of microbeads including the first microbead.

213. The method according to any one of inventive concepts 197-212, wherein:

the hydrogel does not further include a therapeutic agent, and

implanting the piece includes implanting the piece of the hydrogel that does not further include a therapeutic agent.

214. The method according to any one of inventive concepts 197-213, wherein:

the glucose is in the form of a cyclodextrin (CD); and

implanting the hydrogel includes implanting the hydrogel in which the glucose is in the form of the CD.

215. The method according to any one of inventive concepts 197-214, wherein:

the piece of the hydrogel includes a plurality of spaces that extend from outside of the piece into the piece, and

implanting the piece includes implanting the piece that includes the plurality of spaces.

216. The method according to any one of inventive concepts 197-215, wherein:

the hydrogel includes a solid network and a liquid component, and the glucose is part of the solid network; and

implanting the hydrogel includes implanting the hydrogel in which the glucose is part of the solid network.

217. The method according to inventive concept 216, wherein:

the hydrogel is a polypseudorotaxane hydrogel (PPRh) that includes crosslinked axles, and molecules of a cyclodextrin (CD) threaded onto the axles, and

implanting the hydrogel includes implanting the PPRh that includes the crosslinked axles, and the molecules of the cyclodextrin (CD) threaded onto the axles.

218. The method according to inventive concept 217, wherein:

the CD is alpha-CD; and

implanting the PPRh includes implanting the PPRh that includes the molecules of alpha-CD threaded onto the axles.

219. The method according to inventive concept 217, wherein:

each of the axles is an axle of a polymer including ethylene oxide (EO), and

implanting the PPRh includes implanting the PPRh that includes the axles of the polymer including EO.

220. The method according to inventive concept 197, wherein:

the piece contains cells suspended in the hydrogel, and

implanting the piece includes implanting the piece that contains the cells suspended in the hydrogel.

221. The method according to inventive concept 220, wherein:

the cells are mesenchymal stem cells (MSC), and

implanting the piece includes implanting the piece that contains the MSC suspended in the hydrogel.

222. The method according to any one of inventive concepts 197-221, further including identifying the CNS site in response to imaging data.
223. The method according to inventive concept 222, further including generating the imaging data using an imaging technique.
224. The method according to inventive concept 222, further including shaping the piece in response to the imaging data.
225. The method according to inventive concept 224, wherein shaping the piece includes sculpting the piece.
226. The method according to inventive concept 224, wherein shaping the piece includes 3D-printing the piece.
227. Apparatus including:

an implant, including a piece of a hydrogel, the hydrogel including a solid network and a liquid component, wherein the hydrogel includes glucose as a component of the solid network; and

a delivery tool, configured to deliver the implant to a Central Nervous System (CNS) site of a subject.

228. The apparatus according to inventive concept 227, wherein the piece is a macroscopic monolithic piece of the hydrogel.
229. The apparatus according to inventive concept 227, wherein the hydrogel does not further include a therapeutic agent.
230. The apparatus according to inventive concept 227, wherein the hydrogel further includes one or more cell-growth nutrients.
231. The apparatus according to inventive concept 227, wherein the hydrogel further includes one or more neurogenesis-promoting growth factors.
232. The apparatus according to inventive concept 227, wherein the piece includes a plurality of spaces that extend from outside of the piece into the piece.
233. The apparatus according to any one of inventive concepts 227-232, wherein the hydrogel has an elastic modulus of 0.5-10 kPa.
234. The apparatus according to inventive concept 233, wherein the hydrogel has an elastic modulus of 0.5-5 kPa.
235. The apparatus according to inventive concept 233, wherein the hydrogel has an elastic modulus of 1-10 kPa.
236. The apparatus according to inventive concept 235, wherein the hydrogel has an elastic modulus of 1-5 kPa.
237. The apparatus according to inventive concept 236, wherein the hydrogel has an elastic modulus of 1-3 kPa.
238. The apparatus according to inventive concept 227, wherein the hydrogel includes L-lysine.
239. The apparatus according to inventive concept 238, wherein the hydrogel includes a solid network and a liquid component, and the L-lysine is part of the solid network.
240. The apparatus according to inventive concept 238, wherein the hydrogel includes a solid network and a liquid component, and the L-lysine is part of the liquid component.
241. The apparatus according to any one of inventive concepts 227-240, wherein:

the implant is dimensioned to be implanted between a skull and a brain of the subject, and

the delivery tool is configured to deliver the implant to between the skull and the brain of the subject.

242. The apparatus according to any one of inventive concepts 227-240, wherein:

the implant is dimensioned to be implanted within a sulcus of a brain of the subject, and the delivery tool is configured to deliver the implant to within the sulcus.

243. The apparatus according to any one of inventive concepts 227-240, wherein:

the implant is dimensioned to be implanted within a longitudinal fissure of a brain of the subject, and

the delivery tool is configured to deliver the implant to within the longitudinal fissure.

244. The apparatus according to any one of inventive concepts 227-240, wherein:

the implant is dimensioned to be implanted within a corpus callosum of a brain of the subject, and

the delivery tool is configured to deliver the implant to within the corpus callosum.

245. The apparatus according to any one of inventive concepts 227-240, wherein:

the implant is dimensioned to be implanted within a cerebrum of a brain of the subject, and

the delivery tool is configured to deliver the implant to within the cerebrum.

246. The apparatus according to inventive concept 245, wherein:

the implant is dimensioned to be implanted within a cerebral cortex of a brain of the subject, and

the delivery tool is configured to deliver the implant to within the cerebral cortex.

247. The apparatus according to inventive concept 245, wherein:

the implant is dimensioned to be implanted within a hippocampus of a brain of the subject, and

the delivery tool is configured to deliver the implant to within the hippocampus.

248. The apparatus according to inventive concept 245, wherein:

the implant is dimensioned to be implanted within a basal ganglia of a brain of the subject, and

the delivery tool is configured to deliver the implant to within the basal ganglia.

249. The apparatus according to inventive concept 245, wherein:

the implant is dimensioned to be implanted within an olfactory bulb of a brain of the subject, and

the delivery tool is configured to deliver the implant to within the olfactory bulb.

250. The apparatus according to inventive concept 245, wherein:

the implant is dimensioned to be implanted within a frontal lobe of a brain of the subject, and

the delivery tool is configured to deliver the implant to within the frontal lobe.

251. The apparatus according to inventive concept 245, wherein:

the implant is dimensioned to be implanted within a parietal lobe of a brain of the subject, and

the delivery tool is configured to deliver the implant to within the parietal lobe.

252. The apparatus according to inventive concept 245, wherein:

the implant is dimensioned to be implanted within a temporal lobe of a brain of the subject, and

the delivery tool is configured to deliver the implant to within the temporal lobe.

253. The apparatus according to inventive concept 245, wherein:

the implant is dimensioned to be implanted within an occipital lobe of a brain of the subject, and

the delivery tool is configured to deliver the implant to within the occipital lobe.

254. The apparatus according to any one of inventive concepts 227-240, wherein:

the implant is dimensioned to be implanted within a diencephalon of a brain of the subject, and

the delivery tool is configured to deliver the implant to within the diencephalon.

255. The apparatus according to any one of inventive concepts 227-240, wherein:

the implant is dimensioned to be implanted within a mesencephalon of a brain of the subject, and

the delivery tool is configured to deliver the implant to within the mesencephalon.

256. The apparatus according to any one of inventive concepts 227-240, wherein:

the implant is dimensioned to be implanted within a cerebellum of a brain of the subject, and

the delivery tool is configured to deliver the implant to within the cerebellum.

257. The apparatus according to any one of inventive concepts 227-240, wherein:

the implant is dimensioned to be implanted within a pons of a brain of the subject, and the delivery tool is configured to deliver the implant to within the pons.

258. The apparatus according to any one of inventive concepts 227-240, wherein:

the implant is dimensioned to be implanted within a medulla oblongata of a brain of the subject, and

the delivery tool is configured to deliver the implant to within the medulla oblongata.

259. The apparatus according to any one of inventive concepts 227-240, wherein the delivery tool is a transcranial delivery tool, configured to deliver the implant transcranially to a brain of a subject.
260. The apparatus according to any one of inventive concepts 227-240, wherein:

the implant is dimensioned to be implanted within a spinal cord of the subject, and

the delivery tool is configured to deliver the implant to within the spinal cord.

261. The apparatus according to inventive concept 260, wherein:

the implant is dimensioned to be implanted within an epidural space of the spinal cord of the subject, and

the delivery tool is configured to deliver the implant to within the epidural space.

262. The apparatus according to any one of inventive concepts 227-240, wherein the piece is a patch, dimensioned to be implanted between a skull and a brain of the subject.
263. The apparatus according to inventive concept 262, wherein the patch is dimensioned to be implanted in a subarachnoid space of the subject.
264. The apparatus according to inventive concept 262, wherein the patch is dimensioned to be implanted in a subdural space of the subject.
265. The apparatus according to any one of inventive concepts 227-264, wherein the hydrogel is a polypseudorotaxane hydrogel (PPRh) that includes crosslinked axles of a polymer, and molecules of a cyclodextrin (CD) threaded onto the axles.
266. The apparatus according to inventive concept 265, wherein the polymer includes ethylene oxide (EO).
267. The apparatus according to inventive concept 266, wherein the polymer is a homopolymer of EO.
268. The apparatus according to inventive concept 266, wherein the polymer is a copolymer that further includes propylene oxide (PO).
269. The apparatus according to inventive concept 268, wherein the copolymer is a block copolymer including (poly)ethylene oxide (PEO) and (poly)propylene oxide (PPO).
270. The apparatus according to inventive concept 269, wherein the block copolymer is a poloxamer.
271. The apparatus according to inventive concept 265, wherein the molecules of the CD are molecules of alpha-cyclodextrin (alpha-CD).
272. The apparatus according to inventive concept 265, wherein the molecules of the CD are molecules of beta-cyclodextrin (beta-CD).
273. The apparatus according to inventive concept 265, wherein the molecules of the CD are molecules of gamma-cyclodextrin (gamma-CD).
274. The apparatus according to inventive concept 227, wherein the piece contains cells suspended in the hydrogel.
275. The apparatus according to inventive concept 274, wherein the cells are mesenchymal stem cells (MSC).
276. Apparatus including:

an implant, including a piece of a polypseudorotaxane hydrogel (PPRh) that includes:
crosslinked axles of a polymer, and
molecules of a cyclodextrin (CD), threaded onto the axles; and

a delivery tool, configured to deliver the implant to a Central Nervous System (CNS) site of a subject.

277. The apparatus according to inventive concept 276, wherein the piece is a macroscopic monolithic piece of the PPRh.
278. The apparatus according to inventive concept 276, wherein the molecules of the CD are molecules of alpha-cyclodextrin (alpha-CD).
279. The apparatus according to inventive concept 276, wherein the molecules of the CD are molecules of beta-cyclodextrin (beta-CD).
280. The apparatus according to inventive concept 276, wherein the molecules of the CD are molecules of gamma-cyclodextrin (gamma-CD).
281. The apparatus according to inventive concept 276, wherein the PPRh does not further include a therapeutic agent.
282. The apparatus according to inventive concept 276, wherein the implant does not further include a therapeutic agent.
283. The apparatus according to inventive concept 276, wherein the PPRh further includes one or more cell-growth nutrients.
284. The apparatus according to inventive concept 276, wherein the PPRh further includes one or more neurogenesis-promoting growth factors.
285. The apparatus according to inventive concept 276, wherein the piece includes a plurality of spaces that extend from outside of the piece into the piece.
286. The apparatus according to inventive concept 276, wherein the hydrogel has an elastic modulus of 0.5-10 kPa.
287. The apparatus according to any one of inventive concepts 276-286, wherein the hydrogel has an elastic modulus of 0.5-5 kPa.
288. The apparatus according to inventive concept 286, wherein the hydrogel has an elastic modulus of 1-10 kPa.
289. The apparatus according to inventive concept 288, wherein the hydrogel has an elastic modulus of 1-5 kPa.
290. The apparatus according to inventive concept 289, wherein the hydrogel has an elastic modulus of 1-3 kPa.
291. The apparatus according to any one of inventive concepts 276-289, wherein the PPRh further includes L-lysine.
292. The apparatus according to inventive concept 291, wherein the PPRh includes a solid network and a liquid component, and the L-lysine is part of the solid network.
293. The apparatus according to inventive concept 291, wherein the PPRh includes a solid network and a liquid component, and the L-lysine is part of the liquid component.
294. The apparatus according to any one of inventive concepts 276-289, wherein:

the implant is dimensioned to be implanted between a skull and a brain of the subject, and

the delivery tool is configured to deliver the implant to between the skull and the brain of the subject.

295. The apparatus according to any one of inventive concepts 276-289, wherein:

the implant is dimensioned to be implanted within a sulcus of a brain of the subject, and the delivery tool is configured to deliver the implant to within the sulcus.

296. The apparatus according to any one of inventive concepts 276-289, wherein:

the implant is dimensioned to be implanted within a longitudinal fissure of a brain of the subject, and

the delivery tool is configured to deliver the implant to within the longitudinal fissure.

297. The apparatus according to any one of inventive concepts 276-289, wherein:

the implant is dimensioned to be implanted within a corpus callosum of a brain of the subject, and

the delivery tool is configured to deliver the implant to within the corpus callosum.

298. The apparatus according to any one of inventive concepts 276-289, wherein:

the implant is dimensioned to be implanted within a cerebrum of a brain of the subject, and

the delivery tool is configured to deliver the implant to within the cerebrum.

299. The apparatus according to inventive concept 298, wherein:

the implant is dimensioned to be implanted within a cerebral cortex of a brain of the subject, and

the delivery tool is configured to deliver the implant to within the cerebral cortex.

300. The apparatus according to inventive concept 298, wherein:

the implant is dimensioned to be implanted within a hippocampus of a brain of the subject, and

the delivery tool is configured to deliver the implant to within the hippocampus.

301. The apparatus according to inventive concept 298, wherein:

the implant is dimensioned to be implanted within a basal ganglia of a brain of the subject, and

the delivery tool is configured to deliver the implant to within the basal ganglia.

302. The apparatus according to inventive concept 298, wherein:

the implant is dimensioned to be implanted within an olfactory bulb of a brain of the subject, and

the delivery tool is configured to deliver the implant to within the olfactory bulb.

303. The apparatus according to inventive concept 298, wherein:

the implant is dimensioned to be implanted within a frontal lobe of a brain of the subject, and

the delivery tool is configured to deliver the implant to within the frontal lobe.

304. The apparatus according to inventive concept 298, wherein:

the implant is dimensioned to be implanted within a parietal lobe of a brain of the subject, and

the delivery tool is configured to deliver the implant to within the parietal lobe.

305. The apparatus according to inventive concept 298, wherein:

the implant is dimensioned to be implanted within a temporal lobe of a brain of the subject, and

the delivery tool is configured to deliver the implant to within the temporal lobe.

306. The apparatus according to inventive concept 298, wherein:

the implant is dimensioned to be implanted within an occipital lobe of a brain of the subject, and

the delivery tool is configured to deliver the implant to within the occipital lobe.

307. The apparatus according to inventive concept 276, wherein:

the implant is dimensioned to be implanted within a diencephalon of a brain of the subject, and

the delivery tool is configured to deliver the implant to within the diencephalon.

308. The apparatus according to any one of inventive concepts 276-289, wherein:

the implant is dimensioned to be implanted within a mesencephalon of a brain of the subject, and

the delivery tool is configured to deliver the implant to within the mesencephalon.

309. The apparatus according to any one of inventive concepts 276-289, wherein:

the implant is dimensioned to be implanted within a cerebellum of a brain of the subject, and

the delivery tool is configured to deliver the implant to within the cerebellum.

310. The apparatus according to any one of inventive concepts 276-289, wherein:

the implant is dimensioned to be implanted within a pons of a brain of the subject, and the delivery tool is configured to deliver the implant to within the pons.

311. The apparatus according to any one of inventive concepts 276-289, wherein:

the implant is dimensioned to be implanted within a medulla oblongata of a brain of the subject, and

the delivery tool is configured to deliver the implant to within the medulla oblongata.

312. The apparatus according to any one of inventive concepts 276-289, wherein the delivery tool is a transcranial delivery tool, configured to deliver the implant transcranially to a brain of a subject.
313. The apparatus according to any one of inventive concepts 276-289, wherein:

the implant is dimensioned to be implanted within a spinal cord of the subject, and

the delivery tool is configured to deliver the implant to within the spinal cord.

314. The apparatus according to inventive concept 313, wherein:

the implant is dimensioned to be implanted within an epidural space of the spinal cord of the subject, and

the delivery tool is configured to deliver the implant to within the epidural space.

315. The apparatus according to any one of inventive concepts 276-289, wherein the piece is a patch, dimensioned to be implanted between a skull and a brain of the subject.
316. The apparatus according to inventive concept 315, wherein the patch is dimensioned to be implanted in a subarachnoid space of the subject.
317. The apparatus according to inventive concept 315, wherein the patch is dimensioned to be implanted in a subdural space of the subject.
318. The apparatus according to any one of inventive concepts 276-317, wherein the polymer includes ethylene oxide (EO).
319. The apparatus according to inventive concept 318, wherein the polymer is a homopolymer of EO.
320. The apparatus according to inventive concept 276, wherein the polymer is a copolymer that further includes propylene oxide (PO).
321. The apparatus according to inventive concept 320, wherein the copolymer is a block copolymer including (poly)ethylene oxide (PEO) and (poly)propylene oxide (PPO).
322. The apparatus according to inventive concept 321, wherein the block copolymer is a poloxamer.
323. The apparatus according to any one of inventive concepts 276-322, wherein the piece contains cells suspended in the PPRh.
324. The apparatus according to inventive concept 323, wherein the cells are mesenchymal stem cells (MSC).
325. A method, including:

identifying a subject as having a pathological Central Nervous System (CNS) condition; and

in response to the identifying, implanting, at a CNS site of the subject, a piece of a hydrogel that includes L-lysine.

326. The method according to inventive concept 325, wherein:

the hydrogel further includes glucose, and

implanting the hydrogel includes implanting the hydrogel that further includes glucose.

327. The method according to inventive concept 325, wherein:

the condition includes astrogliosis, and

identifying the subject includes identifying the subject as having the condition that includes astrogliosis.

328. The method according to inventive concept 325, wherein:

the condition includes neuronal loss, and

identifying the subject includes identifying the subject as having the condition that includes neuronal loss.

329. The method according to inventive concept 325, wherein:

the condition is a glioma, and

identifying the subject includes identifying the subject as having the glioma.

330. The method according to inventive concept 325, wherein:

the condition is a stroke, and

identifying the subject as having the condition includes identifying the subject as having had the stroke.

331. The method according to inventive concept 325, wherein:

the condition is a neurodegenerative disorder, and

identifying the subject as having the condition includes identifying the subject as having the neurodegenerative disorder.

332. The method according to inventive concept 325, wherein:

the condition is amyotrophic lateral sclerosis, and

identifying the subject as having the condition includes identifying the subject as having amyotrophic lateral sclerosis.

333. The method according to inventive concept 325, wherein:

the condition is Parkinson's disease, and

identifying the subject as having the condition includes identifying the subject as having Parkinson's disease.

334. The method according to inventive concept 325, wherein:

the condition is Alzheimer's disease, and

identifying the subject as having the condition includes identifying the subject as having Alzheimer's disease.

335. The method according to inventive concept 325, wherein:

the condition is Huntington's disease, and

identifying the subject as having the condition includes identifying the subject as having Huntington's disease.

336. The method according to any one of inventive concepts 325-335, wherein:

the piece is a macroscopic monolithic piece of the hydrogel, and

implanting the piece includes implanting the macroscopic monolithic piece of the hydrogel.

337. The method according to any one of inventive concepts 325-335, wherein:

the piece is a first microscopic piece of the hydrogel, and

implanting the piece includes implanting a plurality of microscopic pieces of the hydrogel, the plurality of microscopic pieces including the first microscopic piece.

338. The method according to any one of inventive concepts 325-335, wherein:

the piece is a first microbead of the hydrogel, and

implanting the piece includes implanting a plurality of microbeads of the hydrogel, the plurality of microbeads including the first microbead.

339. The method according to any one of inventive concepts 325-338, wherein:

the hydrogel further includes one or more cell-growth nutrients; and implanting the piece includes implanting the piece of the hydrogel that further includes the one or more cell-growth nutrients.

340. The method according to any one of inventive concepts 325-339, wherein:

the hydrogel further includes one or more neurogenesis-promoting growth factors;

and implanting the piece includes implanting the piece of the hydrogel that further includes the one or more neurogenesis-promoting growth factors.

341. The method according to any one of inventive concepts 325-338, wherein:

the hydrogel does not further include a therapeutic agent, and

implanting the piece includes implanting the piece of the hydrogel that does not further include a therapeutic agent.

342. The method according to any one of inventive concepts 325-338, wherein:

the piece of the hydrogel includes a plurality of spaces that extend from outside of the piece into the piece, and

implanting the piece includes implanting the piece that includes the plurality of spaces.

343. The method according to any one of inventive concepts 325-342, wherein implanting the piece includes implanting the piece in contact with grey matter of the CNS of the subject.
344. The method according to any one of inventive concepts 325-342, wherein implanting the piece includes implanting the piece in contact with white matter of the CNS of the subject.
345. The method according to any one of inventive concepts 325-344, wherein:

the hydrogel includes a solid network and a liquid component, and the L-lysine is part of the solid network; and

implanting the hydrogel includes implanting the hydrogel in which the L-lysine is part of the solid network.

346. The method according to any one of inventive concepts 325-344, wherein:

the hydrogel includes a solid network and a liquid component, and the L-lysine is part of the liquid component; and

implanting the hydrogel includes implanting the hydrogel in which the L-lysine is part of the liquid component.

347. The method according to any one of inventive concepts 325-346, wherein:

the hydrogel is a polypseudorotaxane hydrogel (PPRh) that includes crosslinked axles, and molecules of a cyclodextrin (CD) threaded onto the axles, and

implanting the hydrogel includes implanting the PPRh that includes the crosslinked axles, and the molecules of the cyclodextrin (CD) threaded onto the axles.

348. The method according to inventive concept 347, wherein:

the CD is alpha-CD; and

implanting the PPRh includes implanting the PPRh that includes the molecules of alpha-CD threaded onto the axles.

349. The method according to inventive concept 347, wherein:

each of the axles is an axle of a polymer including ethylene oxide (EO), and

implanting the PPRh includes implanting the PPRh that includes the axles of the polymer including EO.

350. The method according to any one of inventive concepts 325- or 336-349, wherein:

the condition is a traumatic CNS injury, and

identifying the subject as having the condition includes identifying the subject as having the traumatic CNS injury.

351. The method according to inventive concept 350, wherein:

the traumatic CNS injury is a traumatic brain injury (TBI), and

identifying the subject as having the traumatic CNS injury includes identifying the subject as having the TBI.

352. The method according to inventive concept 351, wherein:

the TBI is a diffuse brain injury, and

implanting the piece includes implanting the piece between a skull and a brain of the subject.

353. The method according to inventive concept 350, wherein:

the traumatic CNS injury is a focal injury at an injury site, and

implanting the piece includes implanting the piece at the injury site.

354. The method according to inventive concept 350, wherein:

the traumatic CNS injury is a penetrating injury resulting in a cavity, and

implanting the piece includes implanting the piece in the cavity.

355. The method according to inventive concept 350, further including identifying when a traumatic incident that caused the traumatic CNS injury occurred, and wherein implanting the piece includes implanting the piece 0-4 days after the traumatic incident occurred.
356. The method according to any one of inventive concepts 325-355, wherein implanting the piece includes implanting the piece at a brain of the subject.
357. The method according to inventive concept 356, wherein implanting the piece includes implanting the piece within a sulcus of the brain of the subject.
358. The method according to inventive concept 356, wherein implanting the piece includes implanting the piece within a longitudinal fissure of the brain of the subject.
359. The method according to inventive concept 356, wherein implanting the piece includes implanting the piece at a corpus callosum of the brain of the subject.
360. The method according to inventive concept 356, wherein implanting the piece includes implanting the piece at a cerebrum of the brain of the subject.
361. The method according to inventive concept 360, wherein implanting the piece includes implanting the piece at a cerebral cortex of the brain of the subject.
362. The method according to inventive concept 360, wherein implanting the piece includes implanting the piece at a hippocampus of the brain of the subject.
363. The method according to inventive concept 360, wherein implanting the piece includes implanting the piece at a basal ganglia of the brain of the subject.
364. The method according to inventive concept 360, wherein implanting the piece includes implanting the piece at an olfactory bulb of the brain of the subject.
365. The method according to inventive concept 360, wherein implanting the piece includes implanting the piece at a frontal lobe of the brain of the subject.
366. The method according to inventive concept 360, wherein implanting the piece includes implanting the piece at a parietal lobe of the brain of the subject.
367. The method according to inventive concept 360, wherein implanting the piece includes implanting the piece at a temporal lobe of the brain of the subject.
368. The method according to inventive concept 360, wherein implanting the piece includes implanting the piece at an occipital lobe of the brain of the subject.
369. The method according to inventive concept 356, wherein implanting the piece includes implanting the piece at a diencephalon of the brain of the subject.
370. The method according to inventive concept 356, wherein implanting the piece includes implanting the piece at a mesencephalon of the brain of the subject.
371. The method according to inventive concept 356, wherein implanting the piece includes implanting the piece at a cerebellum of the brain of the subject.
372. The method according to inventive concept 356, wherein implanting the piece includes implanting the piece at a pons of the brain of the subject.
373. The method according to inventive concept 356, wherein implanting the piece includes implanting the piece at a medulla oblongata of the brain of the subject.
374. The method according to inventive concept 325, wherein implanting the piece includes implanting the piece in a spinal cord of the subject.
375. The method according to inventive concept 374, wherein implanting the piece includes implanting the piece in an epidural space of the spinal cord of the subject.
376. The method according to any one of inventive concepts 325-375, wherein:

the piece contains cells suspended in the hydrogel, and

implanting the piece includes implanting the piece that contains the cells suspended in the hydrogel.

377. The method according to inventive concept 376, wherein:

the cells are mesenchymal stem cells (MSC), and

implanting the piece includes implanting the piece that contains the MSC suspended in the hydrogel.

378. The method according to any one of inventive concepts 325-377, further including identifying the CNS site in response to imaging data.
379. The method according to inventive concept 378, further including generating the imaging data using an imaging technique.
380. The method according to inventive concept 378, further including shaping the piece in response to the imaging data.
381. The method according to inventive concept 380, wherein shaping the piece includes sculpting the piece.
382. The method according to inventive concept 380, wherein shaping the piece includes 3D-printing the piece.
383. The method according to any one of inventive concepts 325-326 or 336-349, wherein:

the piece is a patch, and

implanting the piece includes implanting the patch between a skull and a brain of the subject.

384. The method according to inventive concept 383, wherein implanting the patch includes implanting the patch in a subarachnoid space of the subject.
385. The method according to inventive concept 383, wherein implanting the patch includes implanting the patch in a subdural space of the subject.
386. A method, including:

identifying a subject as having reactive astrogliosis; and

in response to the identifying, implanting, at a CNS site of the subject, a piece of a hydrogel that includes L-lysine.

387. The method according to inventive concept 386, wherein:

the hydrogel further includes glucose, and

implanting the hydrogel includes implanting the hydrogel that further includes glucose.

388. The method according to any one of inventive concepts 386-387, wherein:

the piece is a macroscopic monolithic piece of the hydrogel, and

implanting the piece includes implanting the macroscopic monolithic piece of the hydrogel.

389. The method according to any one of inventive concepts 386-387, wherein:

the piece is a first microscopic piece of the hydrogel, and

implanting the piece includes implanting a plurality of microscopic pieces of the hydrogel, the plurality of microscopic pieces including the first microscopic piece.

390. The method according to any one of inventive concepts 386-387, wherein:

the piece is a first microbead of the hydrogel, and

implanting the piece includes implanting a plurality of microbeads of the hydrogel, the plurality of microbeads including the first microbead.

391. The method according to any one of inventive concepts 386-390, wherein:

the hydrogel further includes one or more cell-growth nutrients; and

implanting the piece includes implanting the piece of the hydrogel that further includes the one or more cell-growth nutrients.

392. The method according to any one of inventive concepts 386-391, wherein:

the hydrogel further includes one or more neurogenesis-promoting growth factors;

and implanting the piece includes implanting the piece of the hydrogel that further includes the one or more neurogenesis-promoting growth factors.

393. The method according to any one of inventive concepts 386-390, wherein:

the hydrogel does not further include a therapeutic agent, and

implanting the piece includes implanting the piece of the hydrogel that does not further include a therapeutic agent.

394. The method according to any one of inventive concepts 386-393, wherein:

the piece of the hydrogel includes a plurality of spaces that extend from outside of the piece into the piece, and

implanting the piece includes implanting the piece that includes the plurality of spaces.

395. The method according to any one of inventive concepts 386-394, wherein:

the hydrogel includes a solid network and a liquid component, and the L-lysine is part of the solid network; and

implanting the hydrogel includes implanting the hydrogel in which the L-lysine is part of the solid network.

396. The method according to any one of inventive concepts 386-394, wherein:

the hydrogel includes a solid network and a liquid component, and the L-lysine is part of the liquid component, and

implanting the hydrogel includes implanting the hydrogel in which the L-lysine is part of the liquid component.

397. The method according to any one of inventive concepts 386-396, wherein:

the hydrogel is a polypseudorotaxane hydrogel (PPRh) that includes crosslinked axles, and molecules of a cyclodextrin (CD) threaded onto the axles, and

implanting the hydrogel includes implanting the PPRh that includes the crosslinked axles, and the molecules of the cyclodextrin (CD) threaded onto the axles.

398. The method according to inventive concept 397, wherein:

the CD is alpha-CD; and

implanting the PPRh includes implanting the PPRh that includes the molecules of alpha-CD threaded onto the axles.

399. The method according to inventive concept 397, wherein:

each of the axles is an axle of a polymer including ethylene oxide (EO), and

implanting the PPRh includes implanting the PPRh that includes the axles of the polymer including EO.

400. The method according to inventive concept 386, wherein:

the piece contains cells suspended in the hydrogel, and

implanting the piece includes implanting the piece that contains the cells suspended in the hydrogel.

401. The method according to inventive concept 400, wherein:

the cells are mesenchymal stem cells (MSC), and

implanting the piece includes implanting the piece that contains the MSC suspended in the hydrogel.

402. The method according to any one of inventive concepts 386-401, further including identifying the CNS site in response to imaging data.
403. The method according to inventive concept 402, further including generating the imaging data using an imaging technique.
404. The method according to any one of inventive concepts 386-403, further including shaping the piece in response to imaging data.
405. The method according to inventive concept 404, wherein shaping the piece includes sculpting the piece.
406. The method according to inventive concept 404, wherein shaping the piece includes 3D-printing the piece.
407. A method, including:

locating an astrocytic scar within a Central Nervous System (CNS) of a subject; and

in response to the locating, implanting, adjacent to the scar, a piece of a hydrogel that includes L-lysine.

408. The method according to inventive concept 407, wherein:

the hydrogel further includes glucose, and

implanting the hydrogel includes implanting the hydrogel that further includes glucose.

409. The method according to any one of inventive concepts 407-408, wherein:

the piece is a macroscopic monolithic piece of the hydrogel, and

implanting the piece includes implanting the macroscopic monolithic piece of the hydrogel.

410. The method according to any one of inventive concepts 407-408, wherein:

the piece is a first microscopic piece of the hydrogel, and

implanting the piece includes implanting a plurality of microscopic pieces of the hydrogel, the plurality of microscopic pieces including the first microscopic piece.

411. The method according to any one of inventive concepts 407-408, wherein:

the piece is a first microbead of the hydrogel, and

implanting the piece includes implanting a plurality of microbeads of the hydrogel, the plurality of microbeads including the first microbead.

412. The method according to any one of inventive concepts 407-411, wherein:

the hydrogel further includes one or more cell-growth nutrients; and

implanting the piece includes implanting the piece of the hydrogel that further includes the one or more cell-growth nutrients.

413. The method according to any one of inventive concepts 407-412, wherein:

the hydrogel further includes one or more neurogenesis-promoting growth factors;

and implanting the piece includes implanting the piece of the hydrogel that further includes the one or more neurogenesis-promoting growth factors.

414. The method according to any one of inventive concepts 407-411, wherein:

the hydrogel does not further include a therapeutic agent, and

implanting the piece includes implanting the piece of the hydrogel that does not further include a therapeutic agent.

415. The method according to any one of inventive concepts 407-414, wherein:

the piece of the hydrogel includes a plurality of spaces that extend from outside of the piece into the piece, and

implanting the piece includes implanting the piece that includes the plurality of spaces.

416. The method according to any one of inventive concepts 407-415, wherein implanting the piece of the hydrogel adjacent to the scar includes implanting the piece of the hydrogel within 1 mm of the scar.
417. The method according to inventive concept 416, wherein implanting the piece of the hydrogel adjacent to the scar includes implanting the piece of the hydrogel within 100 microns of the scar.
418. The method according to any one of inventive concepts 407-417, wherein:

the hydrogel includes a solid network and a liquid component, and the L-lysine is part of the solid network; and

implanting the hydrogel includes implanting the hydrogel in which the L-lysine is part of the solid network.

419. The method according to any one of inventive concepts 407-417, wherein:

the hydrogel includes a solid network and a liquid component, and the L-lysine is part of the liquid component; and

implanting the hydrogel includes implanting the hydrogel in which the L-lysine is part of the liquid component.

420. The method according to any one of inventive concepts 407-419, wherein:

the hydrogel is a polypseudorotaxane hydrogel (PPRh) that includes crosslinked axles, and molecules of a cyclodextrin (CD) threaded onto the axles, and

implanting the hydrogel includes implanting the PPRh that includes the crosslinked axles, and the molecules of the cyclodextrin (CD) threaded onto the axles.

421. The method according to inventive concept 420, wherein:

the CD is alpha-CD; and

implanting the PPRh includes implanting the PPRh that includes the molecules of alpha-CD threaded onto the axles.

422. The method according to inventive concept 420, wherein:

each of the axles is an axle of a polymer including ethylene oxide (EO), and

implanting the PPRh includes implanting the PPRh that includes the axles of the polymer including EO.

423. The method according to any one of inventive concepts 407-422, wherein:

the piece contains cells suspended in the hydrogel, and

implanting the piece includes implanting the piece that contains the cells suspended in the hydrogel.

424. The method according to inventive concept 423, wherein:

the cells are mesenchymal stem cells (MSC), and

implanting the piece includes implanting the piece that contains the MSC suspended in the hydrogel.

425. The method according to any one of inventive concepts 407-424, further including identifying the CNS site in response to imaging data.
426. The method according to inventive concept 425, further including generating the imaging data using an imaging technique.
427. The method according to inventive concept 425, further including shaping the piece in response to the imaging data.
428. The method according to inventive concept 427, wherein shaping the piece includes sculpting the piece.
429. The method according to inventive concept 427, wherein shaping the piece includes 3D-printing the piece.
430. A method, including:

identifying a subject as having a glioma at a CNS site of the subject; and

in response to the identifying, implanting, at the CNS site, a piece of a hydrogel that includes L-lysine.

431. The method according to inventive concept 430, wherein:

the hydrogel further includes glucose, and

implanting the hydrogel includes implanting the hydrogel that further includes glucose.

432. The method according to any one of inventive concepts 430-431, wherein:

the glioma is an astrocytoma, and

identifying the subject as having the glioma includes identifying the subject as having the astrocytoma.

433. The method according to any one of inventive concepts 430-432, further including treating the glioma prior to implanting the piece of the hydrogel.
434. The method according to inventive concept 433, wherein treating the glioma includes removing at least part of the glioma.
435. The method according to inventive concept 433, wherein treating the glioma includes ablating at least part of the glioma.
436. The method according to any one of inventive concepts 430-435, wherein:

the piece is a macroscopic monolithic piece of the hydrogel, and

implanting the piece includes implanting the macroscopic monolithic piece of the hydrogel.

437. The method according to any one of inventive concepts 430-435, wherein:

the piece is a first microscopic piece of the hydrogel, and

implanting the piece includes implanting a plurality of microscopic pieces of the hydrogel, the plurality of microscopic pieces including the first microscopic piece.

438. The method according to any one of inventive concepts 430-435, wherein:

the piece is a first microbead of the hydrogel, and

implanting the piece includes implanting a plurality of microbeads of the hydrogel, the plurality of microbeads including the first microbead.

439. The method according to any one of inventive concepts 430-438, wherein:

the hydrogel does not further include a therapeutic agent, and

implanting the piece includes implanting the piece of the hydrogel that does not further include a therapeutic agent.

440. The method according to any one of inventive concepts 430-439, wherein:

the piece of the hydrogel includes a plurality of spaces that extend from outside of the piece into the piece, and

implanting the piece includes implanting the piece that includes the plurality of spaces.

441. The method according to any one of inventive concepts 430-440, wherein:

the hydrogel includes a solid network and a liquid component, and the L-lysine is part of the solid network; and

implanting the hydrogel includes implanting the hydrogel in which the L-lysine is part of the solid network.

442. The method according to any one of inventive concepts 430-440, wherein:

the hydrogel includes a solid network and a liquid component, and the L-lysine is part of the liquid component; and

implanting the hydrogel includes implanting the hydrogel in which the L-lysine is part of the liquid component.

443. The method according to any one of inventive concepts 430-442, wherein:

the hydrogel is a polypseudorotaxane hydrogel (PPRh) that includes crosslinked axles, and molecules of a cyclodextrin (CD) threaded onto the axles, and

implanting the hydrogel includes implanting the PPRh that includes the crosslinked axles, and the molecules of the cyclodextrin (CD) threaded onto the axles.

444. The method according to inventive concept 443, wherein:

the CD is alpha-CD; and

implanting the PPRh includes implanting the PPRh that includes the molecules of alpha-CD threaded onto the axles.

445. The method according to inventive concept 443, wherein:

each of the axles is an axle of a polymer including ethylene oxide (EO), and

implanting the PPRh includes implanting the PPRh that includes the axles of the polymer including EO.

446. The method according to any one of inventive concepts 430-445, wherein:

the piece contains cells suspended in the hydrogel, and

implanting the piece includes implanting the piece that contains the cells suspended in the hydrogel.

447. The method according to inventive concept 446, wherein:

the cells are mesenchymal stem cells (MSC), and

implanting the piece includes implanting the piece that contains the MSC suspended in the hydrogel.

448. The method according to any one of inventive concepts 430-447, further including identifying the CNS site in response to imaging data.
449. The method according to inventive concept 448, further including generating the imaging data using an imaging technique.
450. The method according to inventive concept 448, further including shaping the piece in response to the imaging data.
451. The method according to inventive concept 450, wherein shaping the piece includes sculpting the piece.
452. The method according to inventive concept 450, wherein shaping the piece includes 3D-printing the piece.
453. Apparatus including:

an implant, including a piece of a hydrogel that includes L-lysine; and

a delivery tool, configured to deliver the implant to a Central Nervous System (CNS) site of a subject.

454. The apparatus according to inventive concept 453, wherein the hydrogel includes a solid network and a liquid component, and the L-lysine is part of the solid network.
455. The apparatus according to any one of inventive concepts 453-454, wherein the hydrogel includes a solid network and a liquid component, and the L-lysine is part of the liquid component.
456. The apparatus according to any one of inventive concepts 453-455, wherein the piece is a macroscopic monolithic piece of the hydrogel.
457. The apparatus according to any one of inventive concepts 453-456, wherein the hydrogel does not further include a therapeutic agent.
458. The apparatus according to any one of inventive concepts 453-456, wherein the hydrogel further includes one or more cell-growth nutrients.
459. The apparatus according to any one of inventive concepts 453-456, wherein the hydrogel further includes one or more neurogenesis-promoting growth factors.
460. The apparatus according to any one of inventive concepts 453-459, wherein the piece includes a plurality of spaces that extend from outside of the piece into the piece.
461. The apparatus according to any one of inventive concepts 453-460, wherein:

the implant is dimensioned to be implanted between a skull and a brain of the subject, and

the delivery tool is configured to deliver the implant to between the skull and the brain of the subject.

462. The apparatus according to any one of inventive concepts 453-460, wherein:

the implant is dimensioned to be implanted within a sulcus of a brain of the subject, and the delivery tool is configured to deliver the implant to within the sulcus.

463. The apparatus according to any one of inventive concepts 453-460, wherein:

the implant is dimensioned to be implanted within a longitudinal fissure of a brain of the subject, and

the delivery tool is configured to deliver the implant to within the longitudinal fissure.

464. The apparatus according to any one of inventive concepts 453-460, wherein:

the implant is dimensioned to be implanted within a corpus callosum of a brain of the subject, and

the delivery tool is configured to deliver the implant to within the corpus callosum.

465. The apparatus according to any one of inventive concepts 453-460, wherein:

the implant is dimensioned to be implanted within a cerebrum of a brain of the subject, and

the delivery tool is configured to deliver the implant to within the cerebrum.

466. The apparatus according to inventive concept 465, wherein:

the implant is dimensioned to be implanted within a cerebral cortex of a brain of the subject, and

the delivery tool is configured to deliver the implant to within the cerebral cortex.

467. The apparatus according to inventive concept 465, wherein:

the implant is dimensioned to be implanted within a hippocampus of a brain of the subject, and

the delivery tool is configured to deliver the implant to within the hippocampus.

468. The apparatus according to inventive concept 465, wherein:

the implant is dimensioned to be implanted within a basal ganglia of a brain of the subject, and

the delivery tool is configured to deliver the implant to within the basal ganglia.

469. The apparatus according to inventive concept 465, wherein:

the implant is dimensioned to be implanted within an olfactory bulb of a brain of the subject, and

the delivery tool is configured to deliver the implant to within the olfactory bulb.

470. The apparatus according to inventive concept 465, wherein:

the implant is dimensioned to be implanted within a frontal lobe of a brain of the subject, and

the delivery tool is configured to deliver the implant to within the frontal lobe.

471. The apparatus according to inventive concept 465, wherein:

the implant is dimensioned to be implanted within a parietal lobe of a brain of the subject, and

the delivery tool is configured to deliver the implant to within the parietal lobe.

472. The apparatus according to inventive concept 465, wherein:

the implant is dimensioned to be implanted within a temporal lobe of a brain of the subject, and

the delivery tool is configured to deliver the implant to within the temporal lobe.

473. The apparatus according to inventive concept 465, wherein:

the implant is dimensioned to be implanted within an occipital lobe of a brain of the subject, and

the delivery tool is configured to deliver the implant to within the occipital lobe.

474. The apparatus according to any one of inventive concepts 453-460, wherein:

the implant is dimensioned to be implanted within a diencephalon of a brain of the subject, and

the delivery tool is configured to deliver the implant to within the diencephalon.

475. The apparatus according to any one of inventive concepts 453-460, wherein:

the implant is dimensioned to be implanted within a mesencephalon of a brain of the subject, and

the delivery tool is configured to deliver the implant to within the mesencephalon.

476. The apparatus according to any one of inventive concepts 453-460, wherein:

the implant is dimensioned to be implanted within a cerebellum of a brain of the subject, and

the delivery tool is configured to deliver the implant to within the cerebellum.

477. The apparatus according to any one of inventive concepts 453-460, wherein:

the implant is dimensioned to be implanted within a pons of a brain of the subject, and the delivery tool is configured to deliver the implant to within the pons.

478. The apparatus according to any one of inventive concepts 453-460, wherein:

the implant is dimensioned to be implanted within a medulla oblongata of a brain of the subject, and

the delivery tool is configured to deliver the implant to within the medulla oblongata.

479. The apparatus according to any one of inventive concepts 453-460, wherein the delivery tool is a transcranial delivery tool, configured to deliver the implant transcranially to a brain of a subject.
480. The apparatus according to any one of inventive concepts 453-460, wherein:

the implant is dimensioned to be implanted within a spinal cord of the subject, and

the delivery tool is configured to deliver the implant to within the spinal cord.

481. The apparatus according to inventive concept 480, wherein:

the implant is dimensioned to be implanted within an epidural space of the spinal cord of the subject, and

the delivery tool is configured to deliver the implant to within the epidural space.

482. The apparatus according to any one of inventive concepts 453-460, wherein the piece is a patch, dimensioned to be implanted between a skull and a brain of the subject.
483. The apparatus according to inventive concept 482, wherein the patch is dimensioned to be implanted in a subarachnoid space of the subject.
484. The apparatus according to inventive concept 482, wherein the patch is dimensioned to be implanted in a subdural space of the subject.
485. The apparatus according to any one of inventive concepts 453-484, wherein the hydrogel is a polypseudorotaxane hydrogel (PPRh) that includes crosslinked axles of a polymer, and molecules of a cyclodextrin (CD) threaded onto the axles.
486. The apparatus according to inventive concept 485, wherein the polymer includes ethylene oxide (EO).
487. The apparatus according to inventive concept 486, wherein the polymer is a homopolymer of EO.
488. The apparatus according to inventive concept 486, wherein the polymer is a copolymer that further includes propylene oxide (PO).
489. The apparatus according to inventive concept 488, wherein the copolymer is a block copolymer including (poly)ethylene oxide (PEO) and (poly)propylene oxide (PPO).
490. The apparatus according to inventive concept 489, wherein the block copolymer is a poloxamer.
491. The apparatus according to inventive concept 485, wherein the molecules of the CD are molecules of alpha-cyclodextrin (alpha-CD).
492. The apparatus according to inventive concept 485, wherein the molecules of the CD are molecules of beta-cyclodextrin (beta-CD).
493. The apparatus according to inventive concept 485, wherein the molecules of the CD are molecules of gamma-cyclodextrin (gamma-CD).
494. The apparatus according to any one of inventive concepts 453-493, wherein the piece contains cells suspended in the hydrogel.
495. The apparatus according to inventive concept 494, wherein the cells are mesenchymal stem cells (MSC).
496. Apparatus for treating a subject, the apparatus including an implant, the implant including:

a first region, including a low-elasticity polypseudorotaxane hydrogel (PPRh); and

a second region, including a high-elasticity PPRh that has an elastic modulus that is higher than that of the low-elasticity PPRh.

497. The apparatus according to inventive concept 496, wherein the second region at least partly coats the first region.
498. The apparatus according to any one of inventive concepts 496-497, further including a delivery tool, configured to deliver the implant to a Central Nervous System (CNS) site of a subject.
499. The apparatus according to any one of inventive concepts 496-498, further including cells suspended in the low-elasticity PPRh.
500. The apparatus according to inventive concept 499, wherein the cells are mesenchymal stem cells.
501. The apparatus according to any one of inventive concepts 496-500, wherein the low-elasticity PPRh has an elastic modulus of 0.5-2 kPa.
502. The apparatus according to inventive concept 501, wherein the low-elasticity PPRh has an elastic modulus of 0.5-1.5 kPa.
503. The apparatus according to inventive concept 502, wherein the low-elasticity PPRh has an elastic modulus of 0.8-1.2 kPa.
504. The apparatus according to inventive concept 503, wherein the low-elasticity PPRh has an elastic modulus of about 1 kPa.
505. The apparatus according to inventive concept 502, wherein the low-elasticity PPRh has an elastic modulus of 1-1.5 kPa.
506. The apparatus according to inventive concept 505, wherein the low-elasticity PPRh has an elastic modulus of about 1.3 kPa.
507. The apparatus according to inventive concept 501, wherein the low-elasticity PPRh has an elastic modulus of 1-2 kPa.
508. The apparatus according to inventive concept 496, wherein the high-elasticity PPRh has an elastic modulus of 1-10 kPa.
509. The apparatus according to inventive concept 508, wherein the high-elasticity PPRh has an elastic modulus of 1-5 kPa.
510. The apparatus according to inventive concept 509, wherein the high-elasticity PPRh has an elastic modulus of about 3 kPa.
511. The apparatus according to inventive concept 509, wherein the high-elasticity PPRh has an elastic modulus of 1-3 kPa.
512. The apparatus according to inventive concept 511, wherein the high-elasticity PPRh has an elastic modulus of 2-3 kPa.
513. The apparatus according to inventive concept 509, wherein the high-elasticity PPRh has an elastic modulus of 2.5-3.5 kPa.
514. The apparatus according to inventive concept 513, wherein the high-elasticity PPRh has an elastic modulus of 3-3.5 kPa.
515. The apparatus according to inventive concept 514, wherein the high-elasticity PPRh has an elastic modulus of about 3.2 kPa.
516. A method, including:

obtaining a polypseudorotaxane hydrogel (PPRh); and

immersing the PPRh in an aqueous lysine solution.

517. The method according to inventive concept 516, further including, subsequently to the step of immersing, implanting the PPRh in a central nervous system of a subject.
518. The method according to any one of inventive concepts 516-517, wherein the lysine solution is a sterile lysine solution, and immersing the PPRh in the lysine solution includes immersing the PPRh in the sterile lysine solution.
519. The method according to any one of inventive concepts 516-518, wherein the lysine solution is an L-lysine solution, and wherein immersing the PPRh in the lysine solution includes immersing the PPRh in the L-lysine solution.
520. The method according to inventive concept 519, wherein the L-lysine solution is a poly-L-lysine solution, and wherein immersing the PPRh in the L-lysine solution includes immersing the PPRh in the poly-L-lysine solution.
521. The method according to any one of inventive concepts 516-518, wherein the lysine solution is a D-lysine solution, and wherein immersing the PPRh in the lysine solution includes immersing the PPRh in the D-lysine solution.
522. The method according to inventive concept 519, wherein the D-lysine solution is a poly-D-lysine solution, and wherein immersing the PPRh in the D-lysine solution includes immersing the PPRh in the poly-D-lysine solution.
523. Apparatus, including:

a sealed container;

a sterile lysine solution, sealed within the container;

a sterile polypseudorotaxane hydrogel (PPRh), immersed in the lysine solution, and sealed within the container.

524. A method, including:

obtaining a low-elasticity polypseudorotaxane hydrogel (PPRh); and

at least partly coating the low-elasticity PPRh with a high-elasticity PPRh that has an elastic modulus that is higher than that of the low-elasticity PPRh.

525. An implantable hydrogel that includes glucose, for use in the treatment of a pathological Central Nervous System (CNS) condition.
526. The implantable hydrogel according to inventive concept 525, wherein the hydrogel is a macroscopic monolithic piece of the hydrogel.
527. The implantable hydrogel according to inventive concept 525, wherein the hydrogel is a plurality of microscopic pieces of the hydrogel.
528. The implantable hydrogel according to any one of inventive concepts 525-527, wherein the hydrogel further includes one or more cell-growth nutrients.
529. The implantable hydrogel according to any one of inventive concepts 525-528, wherein the hydrogel further includes one or more neurogenesis-promoting growth factors.
530. The implantable hydrogel according to any one of inventive concepts 525-527, wherein the hydrogel does not further include a therapeutic agent.
531. The implantable hydrogel according to any one of inventive concepts 525-530, wherein the hydrogel is shaped to define a plurality of spaces that extend from outside of the hydrogel into the hydrogel.
532. The implantable hydrogel according to any one of inventive concepts 525-531, wherein the hydrogel has an elastic modulus of 0.5-10 kPa.
533. The implantable hydrogel according to inventive concept 532, wherein the hydrogel has an elastic modulus of 0.5-5 kPa.
534. The implantable hydrogel according to inventive concept 532, wherein the hydrogel has an elastic modulus of 1-10 kPa.
535. The implantable hydrogel according to inventive concept 534, wherein the hydrogel has an elastic modulus of 1-5 kPa.
536. The implantable hydrogel according to inventive concept 535, wherein the hydrogel has an elastic modulus of 1-3 kPa.
537. The implantable hydrogel, according to any one of inventive concepts 525-536, wherein the hydrogel further includes L-lysine.
538. The implantable hydrogel according to inventive concept 537, wherein the hydrogel includes a solid network and a liquid component, and the L-lysine is part of the solid network.
539. The implantable hydrogel according to inventive concept 537, wherein the hydrogel includes a solid network and a liquid component, and the L-lysine is part of the liquid component.
540. The implantable hydrogel according to any one of inventive concepts 525-539, wherein the hydrogel is a polypseudorotaxane hydrogel (PPRh) that includes crosslinked axles of a polymer, and molecules of a cyclodextrin (CD) threaded onto the axles.
541. The implantable hydrogel according to inventive concept 540, wherein the polymer includes ethylene oxide (EO).
542. The implantable hydrogel according to inventive concept 541, wherein the polymer is a homopolymer of EO.
543. The implantable hydrogel according to inventive concept 541, wherein the polymer is a copolymer that further includes propylene oxide (PO).
544. The implantable hydrogel according to inventive concept 543, wherein the copolymer is a block copolymer including (poly)ethylene oxide (PEO) and (poly)propylene oxide (PPO).
545. The implantable hydrogel according to inventive concept 544, wherein the block copolymer is a poloxamer.
546. The implantable hydrogel according to any one of inventive concepts 540-545, wherein the molecules of the CD are molecules of alpha-cyclodextrin (alpha-CD).
547. The implantable hydrogel according to any one of inventive concepts 540-545, wherein the molecules of the CD are molecules of beta-cyclodextrin (beta-CD).
548. The implantable hydrogel according to any one of inventive concepts 540-545, wherein the molecules of the CD are molecules of gamma-cyclodextrin (gamma-CD).
549. The implantable hydrogel according to any one of inventive concepts 525-548, wherein the hydrogel contains cells suspended in the hydrogel.
550. The implantable hydrogel according to inventive concept 549, wherein the cells are mesenchymal stem cells (MSC).
551. The implantable hydrogel according to any one of inventive concepts 525-550, wherein the condition includes astrogliosis.
552. The implantable hydrogel according to any one of inventive concepts 525-550, wherein the condition includes neuronal loss.
553. The implantable hydrogel according to any one of inventive concepts 525-550, wherein the condition is a traumatic CNS injury.
554. The implantable hydrogel according to inventive concept 553, wherein the condition is a traumatic brain injury.
555. The implantable hydrogel according to any one of inventive concepts 525-550, wherein the condition is a glioma.
556. The implantable hydrogel according to any one of inventive concepts 525-550, wherein the condition is a stroke.
557. The implantable hydrogel according to any one of inventive concepts 525-550, wherein the condition is a neurodegenerative disorder.
558. The implantable hydrogel according to any one of inventive concepts 525-550, wherein the condition is amyotrophic lateral sclerosis.
559. The implantable hydrogel according to any one of inventive concepts 525-550, wherein the condition is Parkinson's disease.
560. The implantable hydrogel according to any one of inventive concepts 525-550, wherein the condition is Alzheimer's disease.
561. The implantable hydrogel according to any one of inventive concepts 525-550, wherein the condition is Huntington's disease.
562. An implantable hydrogel that includes L-lysine, for use in the treatment of a pathological Central Nervous System (CNS) condition.
563. The implantable hydrogel according to inventive concept 562, wherein the hydrogel includes a solid network and a liquid component, and the L-lysine is part of the solid network.
564. The implantable hydrogel according to inventive concept 562, wherein the hydrogel includes a solid network and a liquid component, and the L-lysine is part of the liquid component.
565. The implantable hydrogel according to any one of inventive concepts 562-564, wherein the hydrogel further includes glucose.
566. The implantable hydrogel according to any one of inventive concepts 562-565, wherein the hydrogel is a macroscopic monolithic piece of the hydrogel.
567. The implantable hydrogel according to any one of inventive concepts 562-565, wherein the hydrogel is a plurality of microscopic pieces of the hydrogel.
568. The implantable hydrogel according to any one of inventive concepts 562-567, wherein the hydrogel further includes one or more cell-growth nutrients.
569. The implantable hydrogel according to any one of inventive concepts 562-568, wherein the hydrogel further includes one or more neurogenesis-promoting growth factors.
570. The implantable hydrogel according to any one of inventive concepts 562-567, wherein the hydrogel does not further include a therapeutic agent.
571. The implantable hydrogel according to any one of inventive concepts 562-570, wherein the hydrogel is shaped to define a plurality of spaces that extend from outside of the hydrogel into the hydrogel.
572. The implantable hydrogel according to any one of inventive concepts 562-571, wherein the hydrogel is a polypseudorotaxane hydrogel (PPRh) that includes crosslinked axles of a polymer, and molecules of a cyclodextrin (CD) threaded onto the axles.
573. The implantable hydrogel according to inventive concept 572, wherein the polymer includes ethylene oxide (EO).
574. The implantable hydrogel according to inventive concept 573, wherein the polymer is a homopolymer of EO.
575. The implantable hydrogel according to inventive concept 573, wherein the polymer is a copolymer that further includes propylene oxide (PO).
576. The implantable hydrogel according to inventive concept 575, wherein the copolymer is a block copolymer including (poly)ethylene oxide (PEO) and (poly)propylene oxide (PPO).
577. The implantable hydrogel according to inventive concept 576, wherein the block copolymer is a poloxamer.
578. The implantable hydrogel according to any one of inventive concepts 572-577, wherein the molecules of the CD are molecules of alpha-cyclodextrin (alpha-CD).
579. The implantable hydrogel according to any one of inventive concepts 572-577, wherein the molecules of the CD are molecules of beta-cyclodextrin (beta-CD).
580. The implantable hydrogel according to any one of inventive concepts 572-577, wherein the molecules of the CD are molecules of gamma-cyclodextrin (gamma-CD).
581. The implantable hydrogel according to any one of inventive concepts 562-580, wherein the hydrogel contains cells suspended in the hydrogel.
582. The implantable hydrogel according to inventive concept 581, wherein the cells are mesenchymal stem cells (MSC).
583. The implantable hydrogel according to any one of inventive concepts 562-582, wherein the condition includes astrogliosis.
584. The implantable hydrogel according to any one of inventive concepts 562-582, wherein the condition includes neuronal loss.
585. The implantable hydrogel according to any one of inventive concepts 562-582, wherein the condition is a traumatic CNS injury.
586. The implantable hydrogel according to any one of inventive concepts 562-582, wherein the condition is a traumatic brain injury.
587. The implantable hydrogel according to any one of inventive concepts 562-582, wherein the condition is a glioma.
588. The implantable hydrogel according to any one of inventive concepts 562-582, wherein the condition is a stroke.
589. The implantable hydrogel according to any one of inventive concepts 562-582, wherein the condition is a neurodegenerative disorder.
590. The implantable hydrogel according to any one of inventive concepts 562-582, wherein the condition is amyotrophic lateral sclerosis.
591. The implantable hydrogel according to any one of inventive concepts 562-582, wherein the condition is Parkinson's disease.
592. The implantable hydrogel according to any one of inventive concepts 562-582, wherein the condition is Alzheimer's disease.
593. The implantable hydrogel according to any one of inventive concepts 562-582, wherein the condition is Huntington's disease.

The present invention will be more fully understood from the following detailed description of applications thereof, taken together with the drawings, in which:

### BRIEF DESCRIPTION OF THE DRAWINGS

FIG. 1 is a schematic illustration of a hypothesized effect of implanting, at a CNS injury site of a subject, a bioabsorbable hydrogel that comprises glucose, in accordance with some applications of the invention;

FIG. 2 is a flowchart that schematically illustrates a method for use with the hydrogel, in accordance with some applications of the invention;

FIG. 3 is a flowchart that schematically illustrates a method for use with the hydrogel, in accordance with some applications of the invention;

FIGS. 4A-B are schematic illustrations of a system comprising the hydrogel and a delivery tool, in accordance with some applications of the invention; and

FIGS. 5A-D, 6A-C, 7A-C, 8A-C, 9A-D, 10A-B, 11A-B, 12, and 13A-D show data resulting from in vitro and in vivo experiments to examine the effect of PPRhs on cells.

### DETAILED DESCRIPTION OF EMBODIMENTS

Some applications of the invention are based upon the hypothesis, of the inventor, that the presence of a bioabsorbable or biodegradable hydrogel, implanted at a CNS injury site, will promote neuronal growth and/or inhibit astrogliosis.

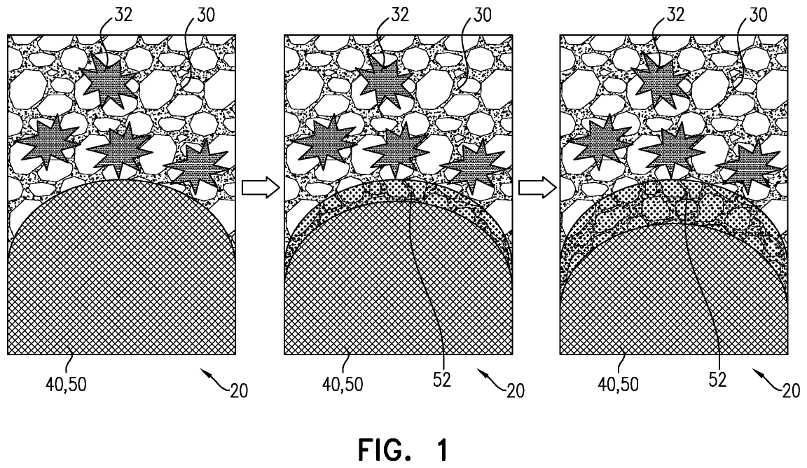

Reference is made to FIG. 1, which is a schematic illustration of this hypothesized effect at a CNS injury site 20 of a subject, in accordance with some applications of the invention. In the first frame, a piece of a bioabsorbable hydrogel 50 that comprises glucose is shown as having been recently implanted at site 20. Endogenous neural cells, including neurons 30 and astrocytes 32, are disposed outside of hydrogel 50. Over time, hydrogel 50 begins to degrade from the surface inwardly, as illustrated in the second and third frames. Reference numeral 52 indicates a partially-degraded region. Hydrogel 50 (e.g., region 52) is hypothesized by the inventor to be a more favorable environment for neurons 30 than for astrocytes 32.

It is hypothesized by the inventor that, for some applications, the degradation of hydrogel 50 further facilitates neurogenesis by releasing the glucose component of the hydrogel, attracting neuronal progenitor cells such as neural stem cells (NSC) and/or promoting their differentiation (Shi, J. et al. (2016) Oncotarget, 7(43), 71052. Dixon, K. J. et al. (2016) Stem cell research, 17(3), 504-513.).

It is hypothesized by the inventor that, for some applications, hydrogel 50 alternatively or additionally serves as a mechanical and/or biochemical barrier that inhibits the migration of astrocytes 32 into the hydrogel. Furthermore, it has been shown that neurogenesis is facilitated by the removal of local glial cells (Moon et al. (2000) Experimental neurology, 161(1), 49-66).

Hydrogel 50 (e.g., region 52 thereof) is therefore hypothesized by the inventor to provide an environment, at injury site 20, in which neurogenesis may occur in the absence of reactive astrogliosis.

It is hypothesized by the inventor that, for some applications, if hydrogel 50 is implanted adjacent an astrocytic scar that has been formed, the neural stem cells that are attracted to site will breach the scar en route to the hydrogel, possibly breaking up the scar. There is therefore provided, in accordance with an application of the present invention, a method comprising (1) locating an astrocytic scar within the CNS of a subject; and (2) in response to locating the scar, implanting, adjacent to the scar, a piece of a hydrogel that includes glucose (e.g., hydrogel 50). For example, the hydrogel may be implanted within 1 mm of the scar, such as within 100 microns of the scar.

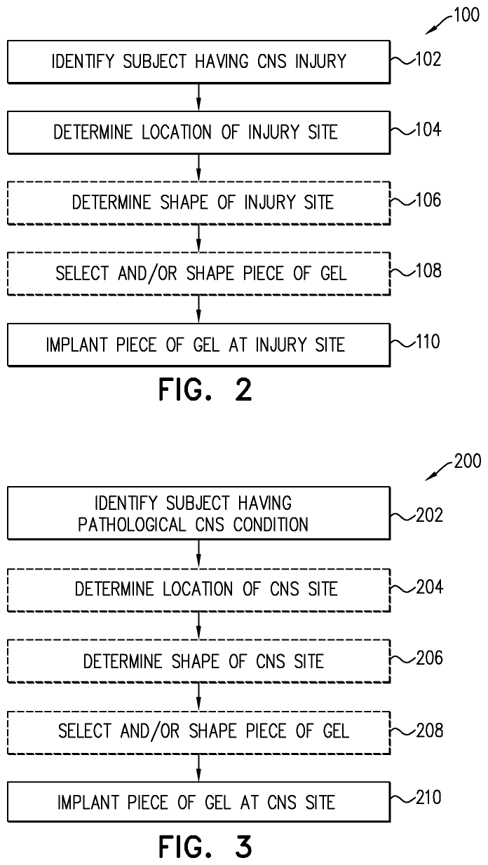

Reference is made to FIG. 2, which is a flowchart that schematically illustrates a method for use with hydrogel 50, in accordance with some applications of the invention. A subject is identified as having a CNS injury, such as traumatic brain injury (TBI) (step 102). In response to this identifying, a piece of hydrogel 50 is implanted at a CNS site of the subject (e.g., at the brain of the subject) (step 110). Typically, the location of the injury site is determined (step 104), and hydrogel 50 is implanted at the identified location.

For some applications, the timing of implantation of hydrogel 50 is important. Therefore, for some such applications, the method comprises identifying when the traumatic incident that caused the traumatic CNS injury occurred, and implanting the hydrogel 0-4 days after the traumatic incident occurred. It is hypothesized that, for some applications, it is advantageous to implant hydrogel 50 as soon as possible, e.g., to prevent post-trauma pathological cellular changes from occurring.

For some applications, the shape of the injury site is determined (e.g., using data from one or more imaging techniques such as MRI, x-ray, CT, or ultrasound) (step 106). In response to the shape of the injury site, an appropriately-shaped piece of hydrogel 50 is selected, and/or a piece of the hydrogel is shaped (e.g., molded, sculpted, or 3D-printed) into an appropriate shape (step 108). For example, for a penetrating injury, hydrogel 50 may be shaped to match (e.g., to substantially fill) the cavity produced by the injury.

For some applications, hydrogel 50 may be considered to be an implant 40 or a component thereof. For some applications, implant 40 consists solely of hydrogel 50. For some applications, implant 40 may comprise both hydrogel 50 and one or more other components, such as a mechanical structural element. For some applications implant 40 and/or hydrogel 50 does not include a therapeutic agent such as a drug or nucleic acid—or at least does not include a therapeutic amount of such a therapeutic agent. For some applications, hydrogel 50 comprises nutrients and/or other factors for promoting neurogenesis within the hydrogel. For some applications, hydrogel 50 only contains the components required to form the hydrogel.

For some applications, the piece of hydrogel 50 that is implanted is a macroscopic monolithic piece of the hydrogel. For example, the piece of the hydrogel may have a greatest dimension that is greater than 0.1 mm and/or less than 50 cm—e.g., between 0.1 mm and 50 cm, such as 0.1-50 mm (e.g., 0.1-30 mm, e.g., 0.1-20 mm, e.g., 0.1-10 mm, e.g., 1-10 mm), 1-10 cm (e.g., 1-5 cm or 5-10 cm), or 10-30 cm (e.g., 10-20 cm, 15-25 cm, or 20-30 cm). For some applications, a plurality of microbeads of hydrogel 50 are implanted, e.g., having a greatest dimension that is 10-1000 microns, e.g., 100-500 microns, such as 100-200 microns. For some applications, a plurality of microscopic pieces of hydrogel 50 are implanted (e.g., having a greatest dimension that is less than 200 microns (e.g., 1-200 microns)).

For some applications, implant 40 contains cells suspended in hydrogel 50. For example, stem cells (e.g., mesenchymal stem cells (MSC)) may be suspended in hydrogel 50. To achieve this, the type of hydrogel used is typically selected such that gelation does not require substances, processes, or temperatures that are noxious to the cells.

For some applications, hydrogel 50 is shaped to define a plurality of spaces that extend from outside of the piece into the piece, e.g., to facilitate controlled cellular migration.

As described hereinabove, pseudorotaxanes with polymer axles comprising poly(ethylene oxide) (PEO) and ring molecules of CD hydrogels have been used to form hydrogels. It is possible to customize characteristics of such hydrogels, such as their viscoelastic properties. Characteristics of the hydrogel may be modified by changing the composition and/or length of the axles. For example, the total number of monomers in each axle and/or the number of ethylene oxide (EO) monomers in each axle may be changed, and other monomers may be included. Alternatively or additionally, characteristics of the hydrogel may be modified by changing the concentration of CD (e.g., the average number of CD molecules threaded onto each axle). Alternatively or additionally, characteristics of the hydrogel may be modified by changing a degree of cross-linking between axles.

It is therefore hypothesized by the inventor that polypseudorotaxane hydrogels (PPRhs) that are based on pseudorotaxanes that comprise CD threaded onto PEO-containing axles, are particularly suited to serve the role of hydrogel 50. While not wishing to be bound to a particular theory or mechanism, it is hypothesized that, for a PPRh, the CD serves as the glucose, and is released as the hydrogel degrades. Characteristics of such hydrogels may be optimized, e.g., as described hereinabove, in order to optimize the effect of implanting the hydrogel. For example, the elasticity, the viscosity, and/or the rate of in vivo degradation of the hydrogel, and/or the rate of CD release from the hydrogel may be optimized.

It has been shown that the behavior (e.g., differentiation) of NSC is affected by mechanical characteristics, such as elasticity, of their substrate (Saha, K. et al. (2008) Biophysical Journal, 95(9), 4426-4438. Kostic, A. et al. (2007) Journal of Cell Science, 120(21), 3895-3904). A further advantage, hypothesized by the inventor, of using a hydrogel whose characteristics are customizable, is that the hydrogel may be optimized to promote neurogenesis by the NSC that arrive at the implantation site.

It is further hypothesized by the inventor that a further advantage to hydrogel 50 being based on pseudorotaxanes that comprise CD threaded onto the axles, is that the glucose component of such a hydrogel is part of the molecular structure of the hydrogel. That is, the glucose is a component of the solid network of the hydrogel (rather than merely being dissolved in the liquid component of the hydrogel). It is hypothesized by the inventor that this facilitates controlled release of the glucose at a rate that is related to the rate of degradation of the hydrogel, e.g., slower than a rate that might occur based on diffusion of the glucose out of the hydrogel.

Whether or not hydrogel 50 is based on pseudorotaxanes that comprise CD threaded onto the axles, the glucose component of hydrogel 50 is typically part of the molecular structure of the hydrogel. However, for some applications, the glucose component of the hydrogel may be dissolved in the liquid component of the hydrogel. For some applications, hydrogel 50 may comprise glucose both as part of the molecular structure of the gel (e.g., CD threaded onto axles), and dissolved in the liquid component of the hydrogel.

For some applications in which hydrogel 50 comprises nutrients and/or other factors for promoting neurogenesis, these components are dissolved in the liquid component of the hydrogel. For some applications, one or more these components are part of the molecular structure of the gel. For some applications, one or more factors that promote neurogenesis are bound (e.g., covalently bound, or ionically bound) to the solid network of the hydrogel (e.g., to the polymer axles).

For some applications in which hydrogel 50 is a PPRh, the polymer that serves as the axle of the pseudorotaxane is a homopolymer (e.g., a PEO homopolymer). For some applications in which hydrogel 50 is a PPRh, the polymer that serves as the axle of the pseudorotaxane is a copolymer comprising EO and propylene oxide (PO). For some such applications, the copolymer is a block copolymer comprising PEO and (poly)propylene oxide (PPO) (also known as polypropylene glycol; PPG). For example, the block copolymer may be a poloxamer, such as Pluronic F68 or Pluronic F127, e.g., as described in Pradal et al. (2013) Biomacromolecules 14, 10, 3780-3792, which is incorporated herein by reference.

For some applications, the CD of the PPRh is alpha-cyclodextrin (alpha-CD). For some applications, the CD of the PPRh is beta-cyclodextrin (beta-CD). For some applications, the CD of the PPRh is gamma-cyclodextrin (gamma-CD). For some applications, the ring molecule of the PPRh is a different cyclic oligosaccharide.

Reference is now made to FIG. 3, which is a flowchart that schematically illustrates a method 200 for use with hydrogel 50, in accordance with some applications of the invention. It is further hypothesized by the inventor that the advantageous effects of hydrogel 50 may be effective in treating a broader range of pathological CNS conditions, especially those that involve neuronal loss and/or pathological astrocytic growth, such as stroke, glioma (e.g., astrocytoma), and neurodegenerative disorders. It is to be noted that reactive astrogliosis is a component in the pathology of some neurodegenerative disorders (Pekny, M. et al. (2014) Physiological reviews, 94(4), 1077-1098). Non-limiting examples of such neurodegenerative disorders include amyotrophic lateral sclerosis (ALS), Huntington's disease, Alzheimer's Disease, and Parkinson's Disease, and it is hypothesized by the inventor that, for some applications, a subject suffering from such a disorder may benefit from implantation of hydrogel 50. It is also to be noted that TBI is associated with the development of certain neurodegenerative disorders later in life (Gavett, B. E. et al. (2010) Alzheimer's research & therapy, 2(3), 18).

Regarding glioma (e.g., astrocytoma), for some applications hydrogel 50 is implanted at (e.g., adjacent to, or surrounding) the glioma. For some applications, at least part (e.g., substantially all) of the glioma is removed or ablated prior to implanting hydrogel 50. For example, hydrogel 50 may be implanted in the space resulting from the removal or ablation of the glioma. It is hypothesized that, for some applications, hydrogel 50 may reduce the likelihood of relapse of the glioma.

Method 200 therefore comprises identifying a subject as having a pathological CNS condition (step 202), and in response to this identifying, implanting a piece of hydrogel 50 at a CNS site (step 210). Similarly to method 100, an appropriate shape and/or implantation site for hydrogel 50 may be facilitated using imaging techniques (steps 204, 206, 208). It is to be noted that for some applications, such as for some neurodegenerative conditions, the shape and/or implantation site may not substantially correspond to the shape and/or location of an anatomical and/or pathological feature.

For some applications, hydrogel 50 is implanted in contact with, or within, grey matter of the CNS. For some applications, hydrogel 50 is implanted in contact with, or within, white matter of the CNS.

For some applications, hydrogel 50 is implanted at the brain of the subject, such as:
within a sulcus of the brain;
within the longitudinal fissure of the brain;
at the corpus callosum of the brain;
at the cerebrum of the brain of the subject (e.g., at the cerebral cortex, at the hippocampus, at the basal ganglia, at the olfactory bulb, at a frontal lobe, at a parietal lobe, at a temporal lobe, or at an occipital lobe);
at the diencephalon of the brain;
at the mesencephalon of the brain;
at the cerebellum of the brain;
at the pons of the brain; or
at a medulla oblongata of the brain.

For some applications, hydrogel 50 is implanted in a spinal cord of the subject, such as in the epidural space.

For some applications, implant 40 and/or hydrogel 50 is shaped as a patch. For some such applications, the patch is implanted between the skull and the brain of the subject, such as in the subarachnoid space or in the subdural space. For some applications, hydrogel 50 (in patch form or otherwise) is implanted between the skull and the brain of the subject in response to identifying a diffuse CNS injury (e.g., a diffuse TBI).

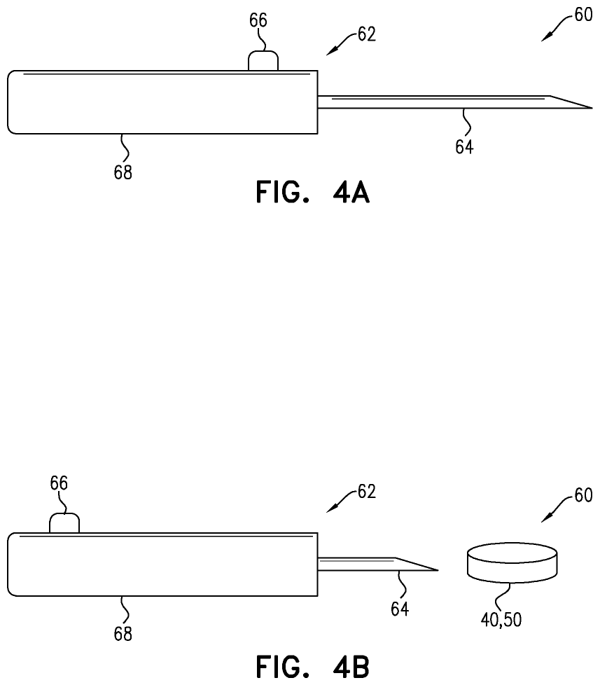

Reference is now made to FIGS. 4A-B, which are schematic illustrations of a system 60, comprising implant 40/hydrogel 50, and a delivery tool 62, in accordance with some applications of the invention. Delivery tool 62 is configured to deliver implant 40/hydrogel 50 to one or more of the CNS sites described hereinabove. For example, delivery tool 62 may be a transcranial delivery tool, configured to deliver the implant/hydrogel transcranially to the brain of a subject.

For some applications, tool 62 comprises a delivery tube (e.g., a hollow needle) 64, and a controller 66, configured to deploy implant 40/hydrogel 50 from the delivery tube. For example, controller 66 may be attached to a handle 68, and moving the controller with respect to the handle deploys the implant/hydrogel, e.g., by sliding delivery tube 64 proximally with respect to the implant/hydrogel. FIG. 4A shows such a tool in a delivery state in which it is advanced into the subject, and FIG. 4B shows the tool in a deployed state, in which implant 40/hydrogel 50 is deployed from delivery tube 64. It is to be noted that the shape of implant 40/hydrogel 50 after deployment is not necessarily the same as the internal shape of delivery tube 64, e.g., due to elasticity or another shape-memory characteristic of the hydrogel. For example, after deployment (e.g., soon after deployment, several minutes after deployment, or several hours after deployment), implant 40/hydrogel 50 may be too large to fit within delivery tube 64.

Reference is again made to FIGS. 1-4B. The PPRh implants described herein are typically sterile, at least at the time of implantation. For example, the PPRh implants may be made under sterile conditions from before the PPRh is formed, or they may be sterilized prior to implantation. The PPRh implants described herein are also typically free of lipopolysaccharide (LPS).

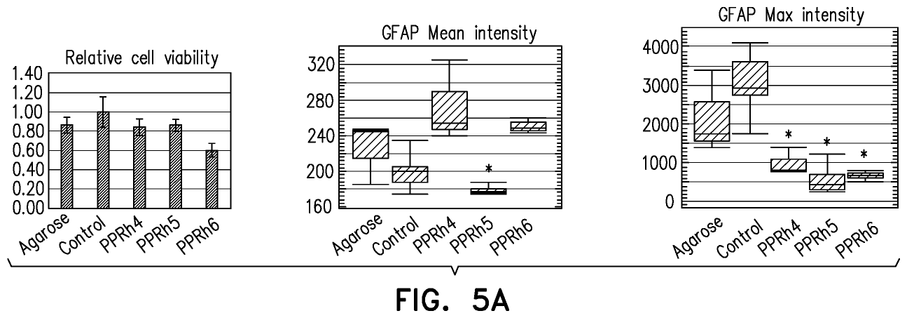

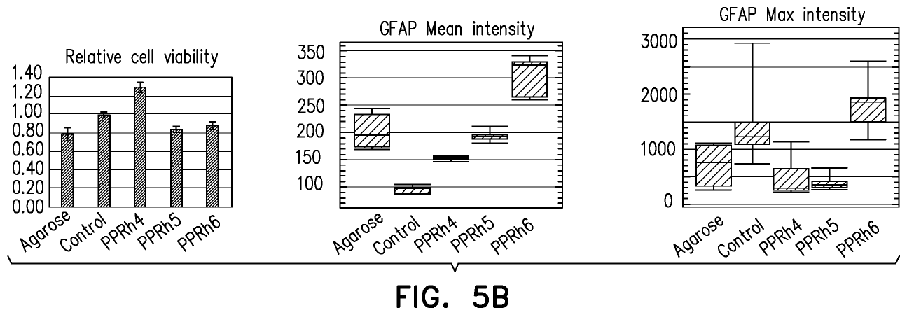

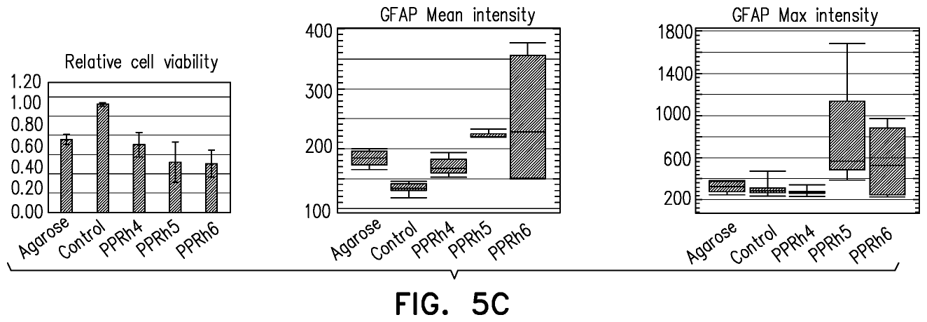

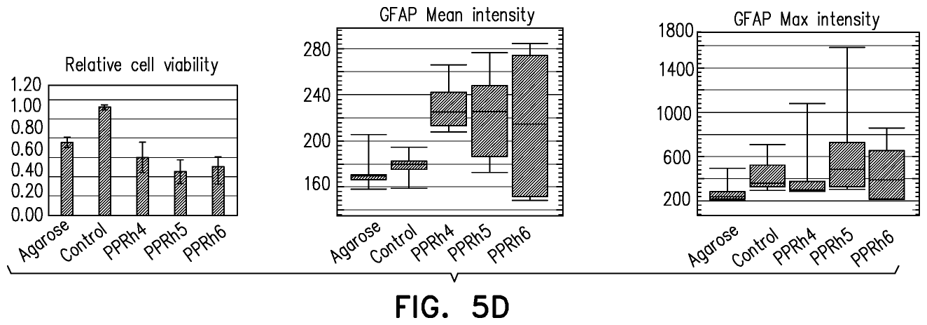

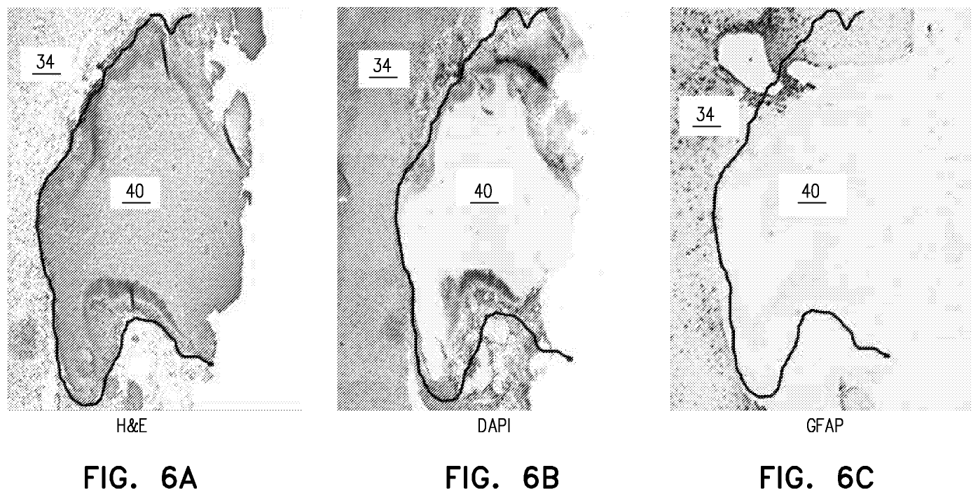

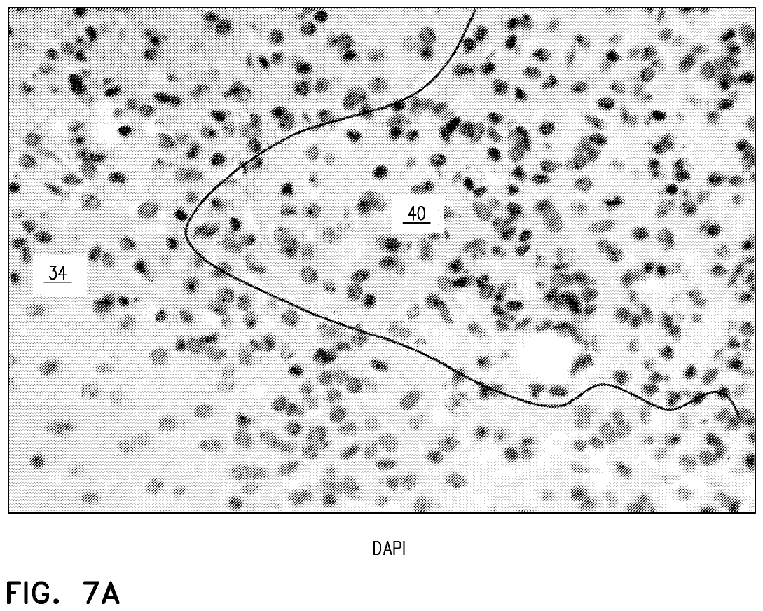

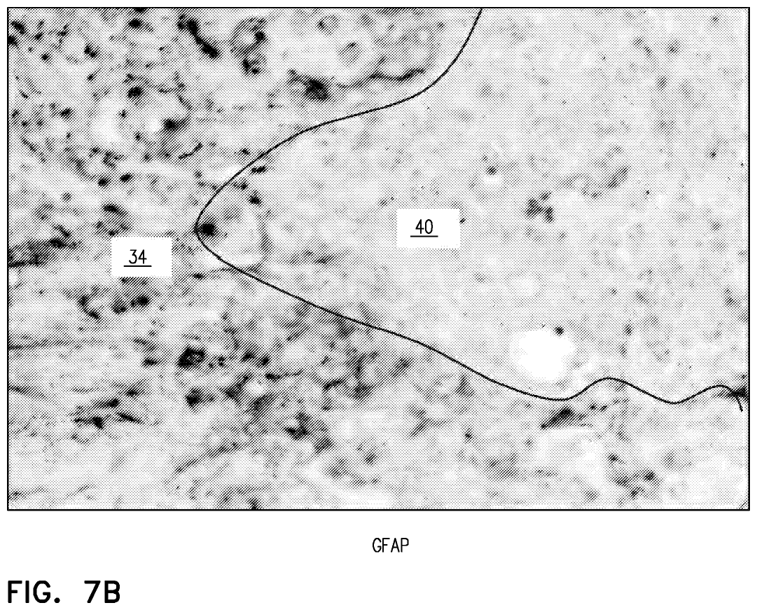

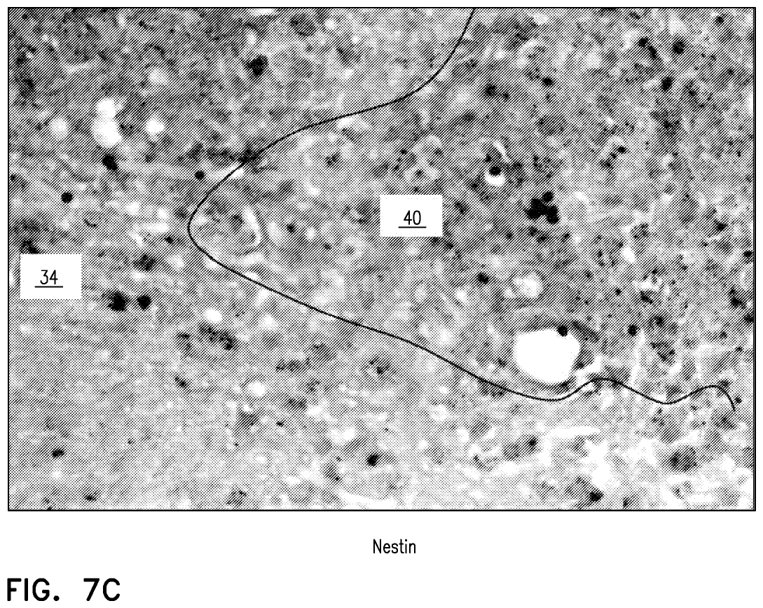

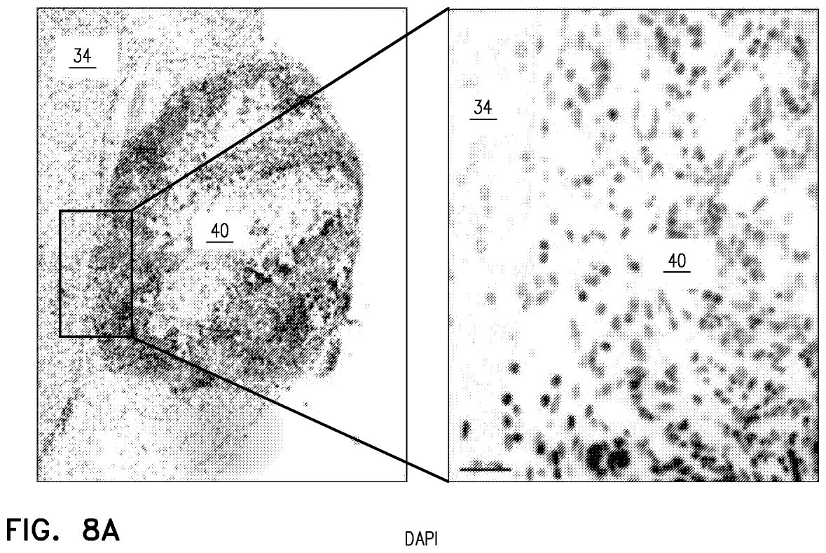

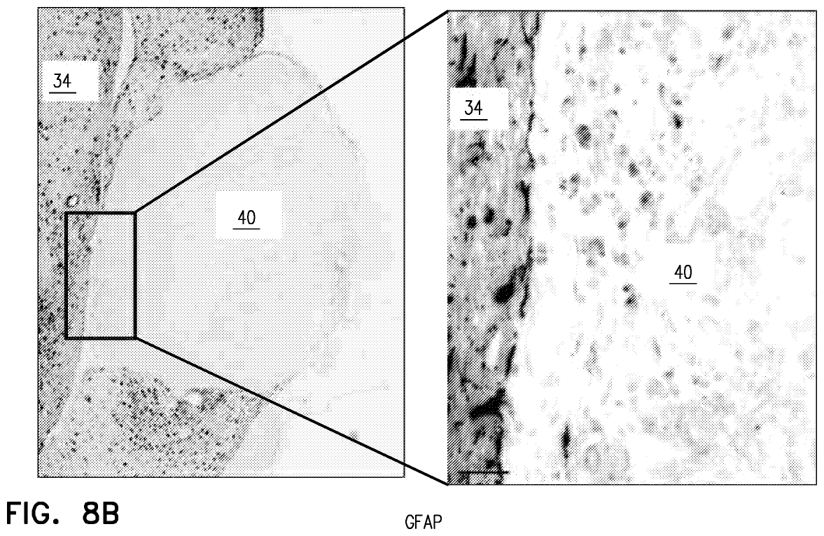

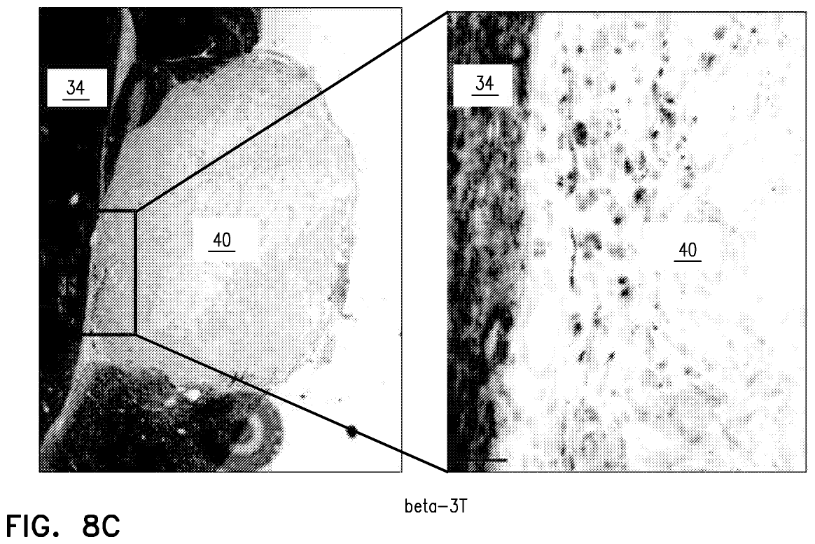

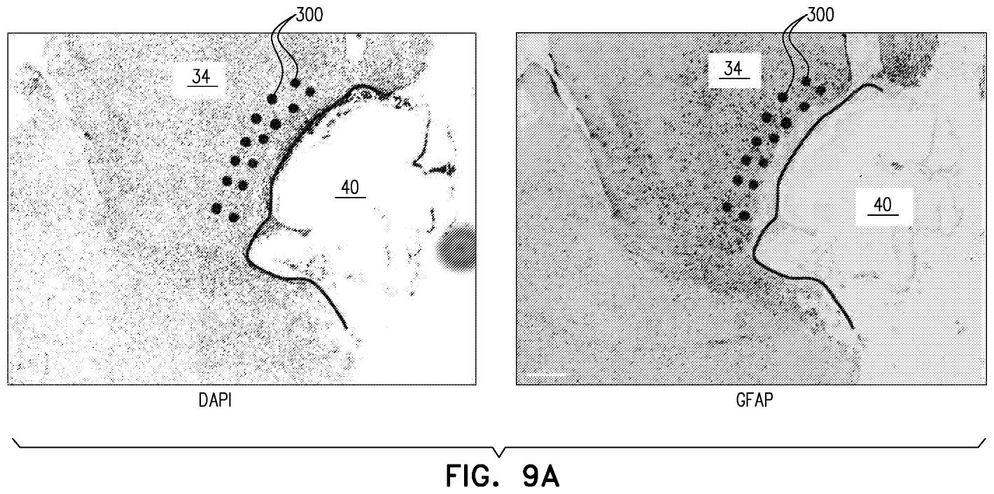

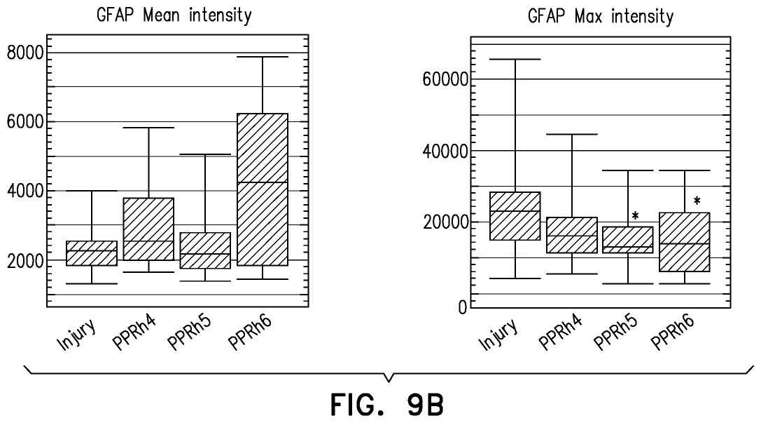

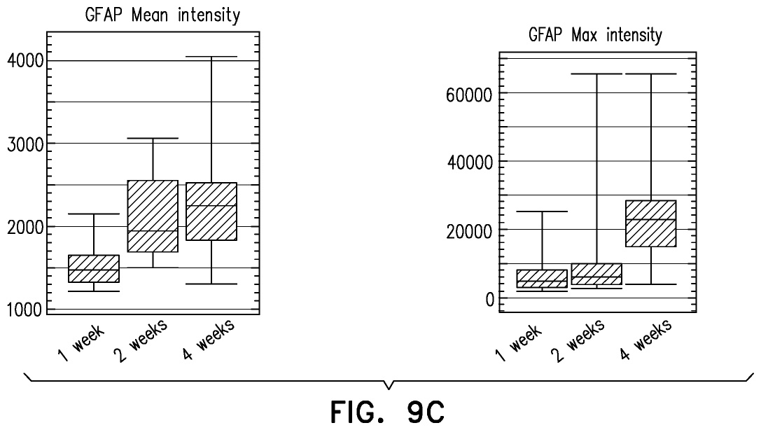

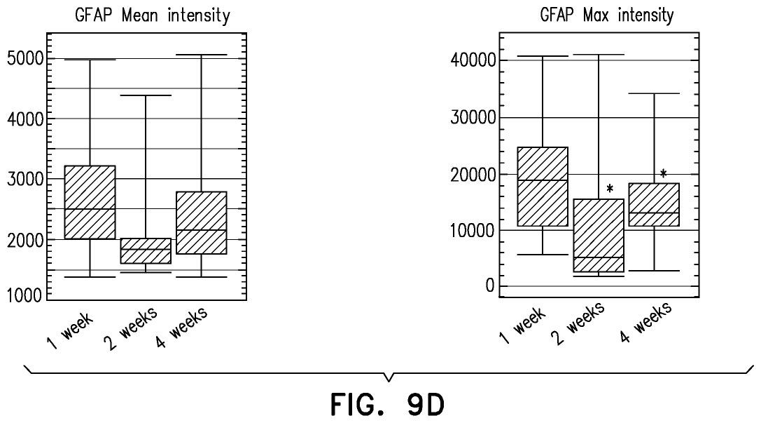

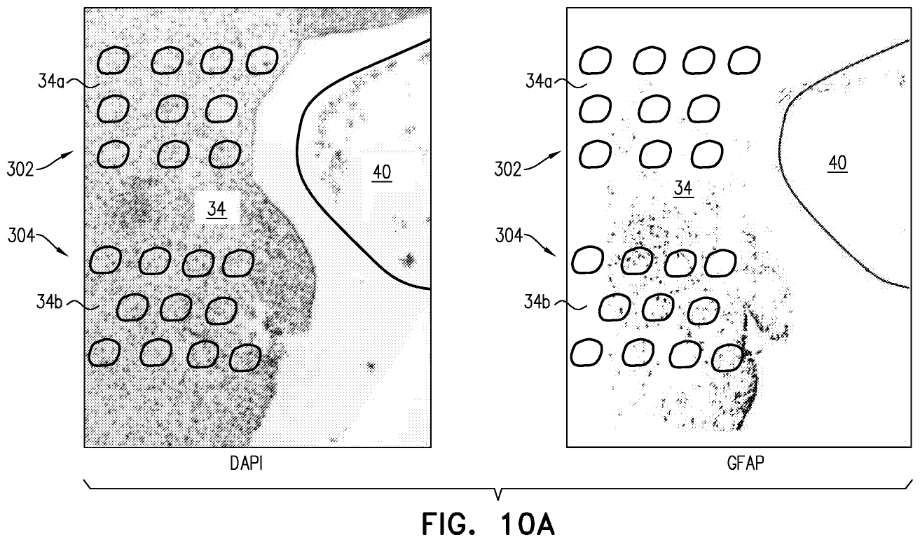

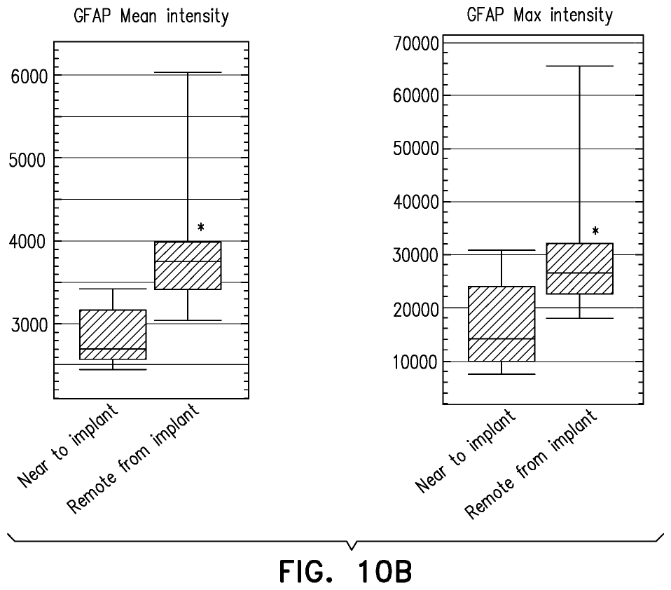

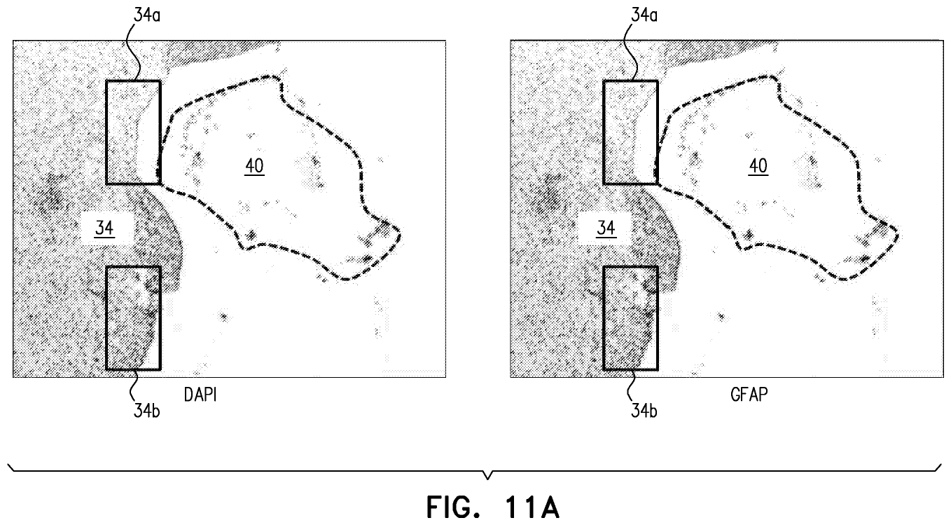

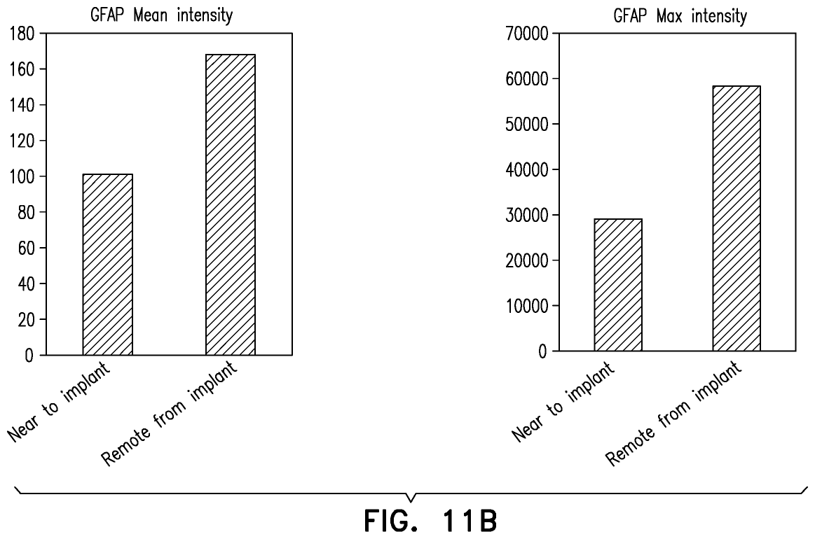

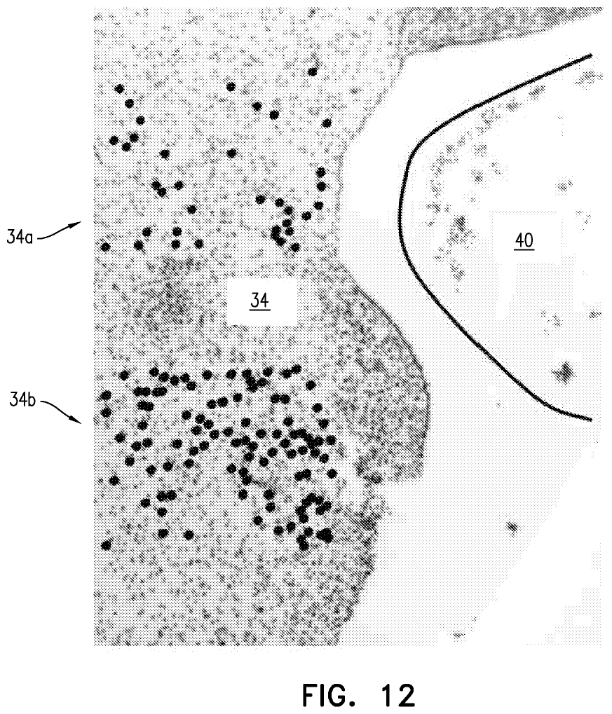

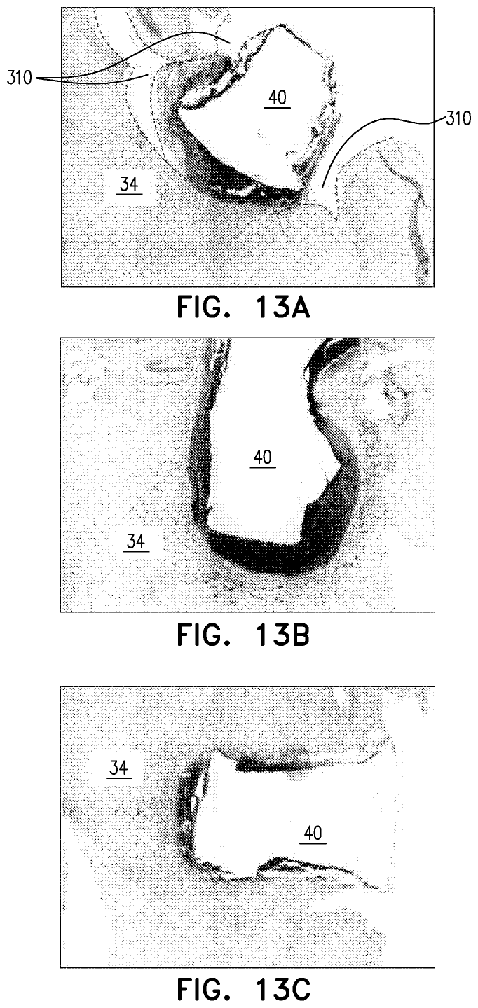

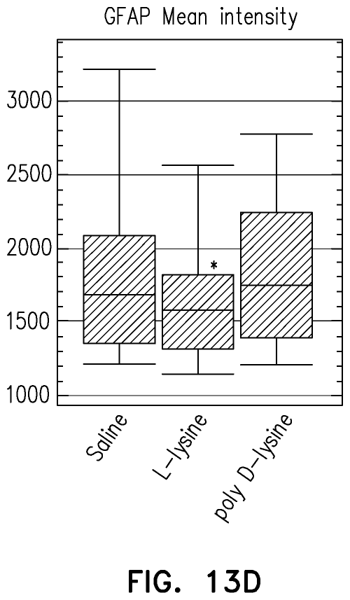

Reference is made to FIGS. 5A-13D, which show data resulting from in vitro and in vivo experiments to examine the effect of PPRhs on cells.

FIGS. 5A-D show data from an experiment in which cells of different types were cultured in vitro, and overlaid with a hydrogel. FIG. 5A shows data for astrocytes, FIG. 5B for neurons, FIG. 5C for Mesenchymal Stem Cells (MSC), and FIG. 5D for A172 cells (a human glioblastoma cell line).

5,000 human placenta-derived MSCs, 5,000 A172 glioblastoma cells, 20,000 primary hippocampal neurons, and 20,000 hippocampal astrocytes from postnatal rat pup brains were seeded, separately, in 96 well plates.

For each cell type, the cells were overlaid with a 1 mm thick disk of one of three PPRhs: PPRh4, PPRh5, or PPRh6 (described hereinbelow). For each cell type a control group was overlaid with a 1 mm thick disk of agarose gel (3 percent). Additionally, for each cell type another control group was provided, in which no hydrogel was laid over the cells.

Each of the PPRhs comprises alpha-CD molecules threaded onto crosslinked axles of PEO homopolymer, each axle being approximately 450 monomers long. PPRh4 has an elastic modulus of about 1.3 kPa. PPRh5 has an elastic modulus of about 3.2 kPa. PPRh6 has an elastic modulus of about 7.2 kPa. Rheological measurements were performed as follows: PPRhs were cut into disks with 20 mm diameter and a 1 mm thickness. Elastic (G′) and loss (G″) moduli were recorded with the frequency increased from 1 rad/s to 100 rad/s and a strain of 0.5 percent on a stress-controlled rheometer (TA instruments, DHR-2) with a 20-mm diameter parallel plate geometry and a measuring gap of 1 mm at room temperature. The averaged values at different frequencies were obtained by averaging the moduli measured from three PPRh-disks with each ratio. All the PPRhs possess frequency-independent elastic moduli.

The different elastic moduli of the various PPRh result from different CD:axle ratios within the PPRh. PPRh4 is made from a starting solution containing 1.45 g alpha-CD, 0.18 g PEO, and 10 ml water. PPRh5 is made from a starting solution containing 2 g alpha-CD, 0.35 g PEO, and 10 ml water. PPRh6 is made from a starting solution containing 2 g alpha-CD, 0.7 g PEO, and 10 ml water.

It is therefore hypothesized that PPRh4 has a CD:PEO ratio by mass of between 7.5:1 and 8.5:1 (e.g., about 8.1:1); PPRh5 has a CD:PEO ratio by mass of between 5:1 and 6:1 (e.g., about 5.7:1); and PPRh6 has a CD:PEO ratio by mass of between 2.5:1 and 3.5:1 (e.g., about 2.9:1).

Other than the differences in their CD:axle ratios, PPRh4, PPRh5, and PPRh6 have the same composition.

Each PPRh disk was immersed in 70 percent ethanol for 30 min, washed 3 times with phosphate-buffered saline (PBS), and soaked for 72 h in growth medium enriched with L-Lysine (20 microgram/ml). Each PPRh disk was then laid onto the seeded cells. For hpMSC and A172, the disk was laid 24 h after the cells were seeded, and for primary neurons and glial cells the disk was laid 6 days after the cells were seeded.

The cells were incubated for 72 h in the presence (or absence) of the corresponding hydrogel.

Subsequently, the cells were examined for viability and expression of glial fibrillary acidic protein (GFAP). GFAP is an intermediate filament protein expressed by several CNS cells, but is expressed particularly strongly by astrocytes. Expression of GFAP is further upregulated in activated astrocytes, and so can serve as an indicator of reactive astrogliosis.

Viability was measured using an XTT-based cell proliferation kit (Biological Industries Israel Beit HaEmek Ltd.; cat 20-300-1000), with the viability of the cells in the control group serving as a reference.

GFAP expression was measured by staining cell cultures with fluorescent anti-GFAP antibody. Stained cultures were tile scanned using Zeiss fluorescence microscope, images were stitched, and GFAP fluorescence was analyzed using Imaris 9.2 software (Bitplane AG, Switzerland). For each image, 3-6 surface segments with similar surface size were positioned within areas where cell population is well established. Surface segments were then quantitatively analyzed for mean and maximal GFAP intensity. Imaris Vantage software was used to compare statistics between groups, and statistical significance was determined by two tail t-test.

From the control groups, mean and maximal GFAP expression were higher in astrocytes than in the other cell types. In astrocytes, all three PPRhs reduced maximal GFAP expression to a statistically significant degree. As described hereinabove, upregulated GFAP expression is associated with activated astrocytes. It is hypothesized by the inventor that reducing maximal expression of GFAP expression may be important in preventing and/or treating astrocyte-mediated pathologies. Interestingly, PPRh5 also reduced mean GFAP expression in astrocytes, whereas PPRh4 and PPRh6 did not. It is hypothesized by the inventor that PPRh5, having an elastic modulus of 2-4 kPa (e.g., 2-3 kPa or 2.5-3.5 kPa, such as 3-3.5 kPa, such as about 3 kPa or about 3.2 kPa) is particularly inhibitive of astrocyte activation, and may therefore be useful in inhibiting reactive astrogliosis in mammalian (e.g., human) subjects.

In all cases except one, the viability of the cultured cells was at least slightly reduced by the presence of any of the gels—agarose or PPRh. However, PPRh4, having an elastic modulus of 1 kPa, promoted the viability of neurons. That is, in the presence of PPRh4, the viability of cultured neurons was higher than in the control. It is hypothesized by the inventor that PPRh4, having an elastic modulus of 0.5-2 kPa (e.g., 1-2 kPa or 0.5-1.5 kPa, such as about 1 kPa or about 1.3 kPa), may be useful in promoting neuronal recovery in mammalian (e.g., human) subjects.

It is therefore hypothesized by the inventor that, for some applications, it is advantageous for the PPRh implant to have a first region (e.g., layer) comprising a low-elasticity PPRh, and a second region (e.g., layer) comprising a high-elasticity PPRh that has an elastic modulus that is higher than that of the low-elasticity PPRh. For some applications, the second region at least partly (e.g., fully) coats the first region. While not wishing to be bound by any particular theory, it is hypothesized by the inventor that, for some such applications, the outer (high-elasticity) region may inhibit astrogliosis and/or formation of an astrocytic scar (e.g., during an earlier period post-implantation), and the inner (low-elasticity) region may promote neuronal recovery (e.g., neurogenesis) (e.g., during a subsequent period post-implantation).

For some applications, the low-elasticity PPRh of the first region has an elastic modulus of 0.5-2 kPa, e.g., 1-2 kPa or 0.5-1.5 kPa, such as 1-1.5 kPa or 0.8-1.2 kPa (e.g., about 1 kPa) or about 1.3 kPa. For some applications, the high-elasticity PPRh of the second region has an elastic modulus of 1-10 kPa, e.g., 1-5 kPa, e.g., 1-3 kPa, 2-3 kPa, 2.5-3.5 kPa, or 3-3.5 kPa (e.g., about 3 kPa or about 3.2 kPa).

There is therefore provided, in accordance with an application of the present invention, a method, comprising (1) obtaining a low-elasticity polypseudorotaxane hydrogel (PPRh); and (2) at least partly coating the low-elasticity PPRh with a high-elasticity PPRh that has an elastic modulus that is higher than that of the low-elasticity PPRh.

As described hereinabove, for some applications, cells such as MSC are included in the PPRh implant, suspended in the hydrogel. For some applications in which the PPRh implant comprises a high-elasticity region and a low-elasticity region, cells (e.g., MSC) are included in the low-elasticity (e.g., inner) region, suspended within the low-elasticity PPRh.

For some applications, e.g., in which the primary desired effect of the implant is to inhibit astrocytes, the PPRh implant to be used comprises primarily (e.g., solely) a high-elasticity PPRh—e.g., having an elastic modulus of 1-10 kPa, e.g., 1-5 kPa, e.g., 1-3 kPa, 2-3 kPa, 2.5-3.5 kPa, or 3-3.5 kPa (e.g., about 3 kPa or about 3.2 kPa). For some applications, e.g., in which the primary desired effect of the implant is to promote neurogenesis, the PPRh implant to be used comprises primarily (e.g., solely) a low-elasticity PPRh—e.g., having an elastic modulus of 0.5-2 kPa, e.g., 1-2 kPa or 0.5-1.5 kPa, such as 0.8-1.2 kPa (e.g., about 1 kPa) or about 1.3 kPa.

Reference is now made to FIGS. 6A-C and 7A-C. An experiment was performed in which a mechanical injury was produced in the brain of a mouse, and one day later, a PPRh implant 40 comprising PPRh5 was implanted.

The mechanical injury was produced as follows: 2-3-month-old Sabra mice were anesthetized by an injection of 100 mg/kg Xylazine/Ketamine into the abdominal cavity. The skull area between Lambda and Bregma was exposed, and two apertures through the skull were formed using a 2.3 mm drill (RWD Life Science, China) until a round skull disk was detached, exposing the brain. The apertures were positioned on opposite sides of the skull, 2 mm from the Bregma towards the Lambda location. A 1.8 mm drill was then used to create a cavity 2 mm deep in the exposed brain tissue, with the drill pointing toward the hippocampus. Skull disks were replaced, and the wound was stitched. After surgery mice received 50 mg/kg Carpofen and 2 ml saline. For mice that received a PPRh implant, a secondary surgery was performed 24 hours after the initial surgery. Each implant comprised a piece of PPRh having a height of 1 mm and a diameter of 1.8 mm. Each implant was immersed for 24 h in saline containing 1 percent L-Lysine (w/v), and then implanted into the right-hand side cavity. Two weeks later, the mice were sacrificed, and sections of brain tissue were stained and examined.

FIGS. 6A-C show such sections of the brain tissue, at the boundary between the tissue 34 itself and the implanted PPRh implant 40. A first section was stained with hematoxylin and eosin (H&E). An adjacent section was stained with (i) DAPI, and (ii) anti-GFAP. FIG. 6A shows visualization of the H&E staining under light microscopy, to visualize cells. FIG. 6B shows visualization of the DAPI staining under fluorescent microscopy, to visualize nucleic acids. FIG. 6C shows visualization of the anti-GFAP staining under fluorescent microscopy, to visualize GFAP expression. The solid line has been superimposed on top of these images, to show the outline of the PPRh implant. FIGS. 6A-B show cellular infiltration into the PPRh implant, while FIG. 6C shows that the infiltrating cells predominantly do not express GFAP.

FIGS. 7A-C show another section of the brain tissue, at the boundary between the tissue 34 itself and the implanted PPRh implant 40. This section was stained with (i) DAPI, (ii) anti-GFAP, and (iii) anti-nestin.

FIG. 7A shows visualization of the DAPI staining under fluorescent microscopy, to visualize nucleic acids. Like FIG. 6B, FIG. 7A shows cellular infiltration into the PPRh implant. FIG. 7B shows visualization of the anti-GFAP staining under fluorescent microscopy, to visualize GFAP expression. Like FIG. 6C, FIG. 7B shows that the infiltrating cells predominantly do not express GFAP. FIG. 7C shows visualization of the anti-nestin staining under fluorescent microscopy, and shows that nestin is expressed both by cells outside of the PPRh implant, and by cells that have infiltrated the PPRh implant. Nestin (neuroectodermal stem cell marker) is an intermediate filament protein, expressed inter alia in neuronal precursor cells. It is therefore hypothesized by the inventor that the cells infiltrating the PPRh implant (comprising PPRh5) were non-astrocytic neuronal precursor cells.

Reference is now made to FIGS. 8A-C. An experiment was performed in which a mechanical injury was produced in the brain of a mouse, and one day later, a PPRh implant 40 comprising PPRh4 was implanted. The mechanical injury was produced as described hereinabove. The PPRh implant was implanted as described hereinabove. Two weeks later, the mice were sacrificed, and sections of brain tissue were stained and examined.

FIGS. 8A-C show a section of the brain tissue, at the boundary between the tissue 34 itself and the implanted PPRh implant 40. This section was stained with (i) DAPI, (ii) anti-GFAP, and (iii) anti-beta 3 tubulin (anti-class III ß-tubulin; anti-beta-3T). Each of FIGS. 8A-C includes a lower-magnification image, and a corresponding higher-magnification enlargement of a region.

FIG. 8A shows visualization of the DAPI staining under fluorescent microscopy, to visualize nucleic acids. Like FIGS. 6B and 7A, FIG. 8A shows cellular infiltration into the PPRh implant. FIG. 8B shows visualization of the anti-GFAP staining under fluorescent microscopy, to visualize GFAP expression. Like FIGS. 6C and 7B, FIG. 8B shows that the infiltrating cells predominantly do not express GFAP. FIG. 8C shows visualization of the anti-beta-3T staining under fluorescent microscopy, and shows that beta-3T is expressed by cells outside of the PPRh implant. Interestingly, FIG. 8C also shows that beta-3T is expressed by some of the cells that have infiltrated the PPRh implant. Beta-3T is a microtubule element expressed by neurons, but not by glial cells such as astrocytes. It is therefore hypothesized by the inventor that the PPRh implant (comprising PPRh4) facilitates, or even promotes, differentiation of cells (e.g., neural stem cells) toward becoming neurons (i.e., neurogenesis) close to and/or within the implant.

In some fluorescent photomicrographs, it was observed that some cells co-expressed GFAP and beta-3T. It is therefore further hypothesized by the inventor that, for some applications, a PPRh implant comping PPRh4 may induce “reprogramming” of astrocytes to become neurons.

Reference is made to FIGS. 9A-D, 10A-B, 11A-B, and 12. An experiment was performed in which a mechanical brain injury was produced in mice, as described hereinabove. One day later, mice in one group received PPRh implants 40 comprising PPRh4, mice in another group received implants comprising PPRh5, and mice in another group received implants comprising PPRh6. Mice in an injury-only control group received no implant. 1 week, 2 weeks, or 4 weeks later, mice were sacrificed, and sections of brain tissue were stained and examined by microscopy. There were four mice in each group at each time point.

FIGS. 9A-D show an analysis performed on the brain tissue, FIGS. 10A-B show another analysis of the brain tissue, FIGS. 11A-B show yet another analysis of the brain tissue, and FIG. 12 shows still another analysis of the brain tissue.

Reference is made to FIGS. 9A-D. Discrete regions of interest (ROI) 300 of the microscopic images were defined using Imaris 9.2 software. Each ROI had a diameter of 256 microns, and was positioned based on where cells were evenly spread and confluence is similar, using DAPI visualization only. FIG. 9A shows DAPI visualization and anti-GFAP visualization of a particular slice of brain tissue, and example ROI are shown as dark circles. As shown in FIG. 9A, these ROI were defined in tissue zones adjacent the injury site. For mice that received a PPRh implant, these tissue zones were therefore also close to the implant. GFAP expression within each of the ROI was quantified using Imaris Vantage software, and results between ROI were compared—this data is shown in FIGS. 9B-C.

FIG. 9B shows data from mice sacrificed at 4 weeks. Although no statistically-significant change in mean GFAP expression was observed, maximal GFAP expression was observed to be reduced in mice that received the PPRh implants. The asterisk indicates p<0.05 (two tail t-test).

FIGS. 9C and 9D shows data from the control and PPRh5 groups, comparing the different timepoints. Whereas GFAP expression (mean and maximal) increased between the 1-week and 4-week timepoints, maximal GFAP expression was reduced at the 2-week and 4-week timepoints, compared to the 1-week timepoint. The asterisk indicates p<0.05 (two tail t-test).

Reference is made to FIGS. 10A-B, which show data from 2 weeks after implantation of a PPRh implant 40 comprising PPRh5. In this analysis, two groups of ROI were defined. A first group 302 included ROI that were within a proximal tissue zone 34a near to the PPRh implant, and a second group 304 included ROI that were within a distal tissue zone 34b remote from the PPRh implant. Both groups included ROI that were shallower (i.e., closer to the injury site), and ROI that were deeper within the tissue. That is, zones 34a and 34b each extended from shallow areas to deeper within tissue 34. GFAP expression within each of the ROI was quantified, and results between ROI were compared.

As shown in FIG. 10B, both mean and maximal GFAP expression were significantly lower (asterisk indicates p<0.05; two tail t-test) in proximal tissue zone 34a compared to in distal tissue zone 34b. That is, proximity to the PPRh implant correlated with reduced GRAP expression.

Reference is made to FIGS. 11A-B, which show data from 2 weeks after implantation of a PPRh implant comprising PPRh5. FIGS. 11A-B show a similar analysis to that of FIGS. 10A-B, comparing a proximal tissue zone 34a with a distal tissue zone 34b, except that rather than defining multiple block-like ROI within each tissue zone, a single filamentous ROI was defined along the surface of the tissue within each tissue zone, using Imaris Filament segmentation. FIG. 11A shows DAPI visualization and anti-GFAP visualization.

As shown in FIG. 11B, both mean and maximal GFAP expression were lower in proximal tissue zone 34a compared to in distal tissue zone 34b. That is, proximity to the PPRh implant correlated with reduced GRAP expression. It is hypothesized by the inventor that the data shown in FIGS. 11A-B indicates an inhibitory effect of the PPRh implant on the formation of astrocytic scars. That is, not only does the PPRh implant appear to inhibit astrogliosis generally, it also appears to inhibit the formation astrocytic scars. As described hereinabove, astrocytic scars may detrimentally inhibit influx of neural stem cells, and it is hypothesized by the inventor that inhibition of scar formation may facilitate neuronal recovery (e.g., neurogenesis) at the injury site.

Reference is now made to FIG. 12. Astrogliosis is mediated by highly-activated astrocytes (Brandao, M. et al. (2018) doi:10.1002/glia.23520; Wanner, I B et al. (2013) doi:10.1523/jneurosci.2121-13.2013). It is therefore hypothesized by the inventor that reducing the number of highly-activated astrocytes in the vicinity of an injury may inhibit astrogliosis.

FIG. 12 shows an analysis in which cells expressing more than a threshold level of GFAP are counted. Again, a proximal tissue zone 34a (proximal to the implant) and a distal tissue zone 34b (distal from the implant) were defined. Tissue zones 34a and 34b were of equal area. Mean GFAP expression across both areas was determined, based on anti-GFAP visualization. A GFAP expression threshold was set at 160 percent of the mean GFAP expression level, and cells expressing GFAP at a level higher than the threshold were counted. Each such cell is indicated in FIG. 12 as a black dot. Within distal tissue zone 34b, 102 such cells were counted, whereas only 36 such cells were counted within proximal tissue zone 34a. That is, not only does the PPRh implant appear to inhibit GFAP expression at a cell population level, the PPRh implant also appears to inhibit individual cells from expressing high levels of GFAP. It is therefore hypothesized by the inventor that the PPRh implant inhibits astrocytes from becoming highly activated.

Reference is now made to FIGS. 13A-D. The inventor frequently observed, in tissue sections, gaps between an implanted PPRh implant and the surrounding tissue. It is hypothesized by the inventor that such gaps may reduce the efficacy of the PPRh implant. It was further hypothesized by the inventor that such gaps may be caused by hydrophobicity of the PPRh, and therefore that increasing the hydrophilicity of the PPRh implant may increase the degree of contact between the implant and the adjacent tissue. It was hypothesized that this might be achieved by including a hydrophilic moiety, such as a hydrophilic amino acid, such as lysine, in the PPRh of the PPRh implant.

FIGS. 13A-C are photomicrographs, showing different PPRh implants, 2 weeks after implantation at respective injury sites. In each case, the PPRh implant comprised PPRh5. For 24 hours prior to implantation, the implants in FIGS. 13A-C were immersed in saline (FIG. 13A), saline containing 1 percent L-lysine (w/v) (FIG. 13B), or saline containing 1 percent poly D-lysine (w/v) (FIG. 13C).

In FIG. 13A, several gaps 310 are visible between the implant and the adjacent tissue. FIGS. 13B and 13C show that the presence of lysine (either L-lysine or poly D-lysine) in the PPRh implant increased the degree of contact between the implant and the adjacent tissue.

FIG. 13D shows mean GFAP expression in tissues adjacent the PPRh implants shown in FIGS. 13A-C. Interestingly, the presence of L-lysine in the PPRh implant significantly increased its inhibition of GFAP compared to the saline-containing control PPRh implant. This effect did not appear to occur with the PPRh implant containing poly D-lysine, for which GFAP mean intensity was similar to that of the saline-containing control PPRh implant.

In the experiments described herein, lysine was introduced into the hydrogel by immersing the hydrogel in a lysine solution, and therefore the hydrogel comprised lysine in the liquid component of the hydrogel. However, it is hypothesized by the inventor that a similar effect may be achieved in a PPRh (or other hydrogel) that comprises lysine in the solid network of the hydrogel. It is further hypothesized that this may extend the duration of the effect, e.g., because the lysine may be retained within the hydrogel for longer.

Therefore, in accordance with some applications of the present invention, hydrogels described herein may include lysine (e.g., L-lysine and/or D-lysine). For some applications, the lysine is monomeric. For some applications, the lysine is polymeric. For some applications, the lysine is included in the liquid component of the hydrogel. For some applications, the lysine is included in the solid network of the hydrogel.

There is further provided, in accordance with an application of the present invention, a method, comprising obtaining a polypseudorotaxane hydrogel (PPRh), and immersing the PPRh in an aqueous lysine solution. For some applications, the lysine solution comprises 0.5-5 percent lysine (w/v) (e.g., 0.5-2 percent lysine (w/v), such as about 1 percent lysine (w/v)). It is to be noted that the scope of the present invention includes the aqueous lysine solution comprising additional components other than lysine and water.

There is further provided, in accordance with an application of the present invention, apparatus, comprising (1) a sealed container; (2) a sterile lysine solution, sealed within the container; and a sterile polypseudorotaxane hydrogel (PPRh), immersed in the lysine solution, and sealed within the container.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. A method, comprising:
identifying a subject as having a pathological Central Nervous System (CNS) condition; and
in response to the identifying, implanting, at a CNS site of the subject, a piece of a polypseudorotaxane hydrogel (PPRh) that has an elastic modulus of 0.5-10 kPa, and includes:
crosslinked axles of a polymer including ethylene oxide (EO), and
molecules of a cyclodextrin (CD), threaded onto the axles.

2. The method according to claim 1, wherein:
the PPRh does not further include a therapeutic agent, and
implanting the piece comprises implanting the piece of the PPRh that does not further include a therapeutic agent.

3. The method according to claim 1, wherein:
the molecules of the CD are molecules of alpha-cyclodextrin (alpha-CD), and
implanting the piece of the PPRh comprises implanting a piece of a PPRh that includes the molecules of alpha-CD, threaded onto the axles.

4. The method according to claim 1, wherein:
the piece of the PPRh includes a plurality of spaces that extend from outside of the piece into the piece, and
implanting the piece comprises implanting the piece that includes the plurality of spaces.

5. The method according to claim 1, wherein:
the PPRh further includes one or more cell-growth nutrients; and
implanting the piece comprises implanting the piece of the PPRh that further includes the one or more cell-growth nutrients.

6. The method according to claim 1, wherein:
the PPRh further includes one or more neurogenesis-promoting growth factors; and
implanting the piece comprises implanting the piece of the PPRh that further includes the one or more neurogenesis-promoting growth factors.

7. The method according to claim 1, wherein:
the polymer is a homopolymer of EO; and
implanting the piece comprises implanting the piece of the PPRh that includes the crosslinked axles of the homopolymer of EO.

8. The method according to claim 1, wherein:
the piece is a macroscopic monolithic piece of the PPRh, and
implanting the piece comprises implanting the macroscopic monolithic piece of the PPRh.

9. The method according to claim 1, wherein:
the piece is a first microscopic piece of the PPRh, and
implanting the piece comprises implanting a plurality of microscopic pieces of the PPRh, the plurality of microscopic pieces including the first microscopic piece.

10. (canceled)

11. The method according to claim 1, wherein:
the polymer is a copolymer that further includes propylene oxide (PO), and
implanting the piece comprises implanting the piece of the PPRh that includes the copolymer that further includes PO.

12. The method according to claim 11, wherein:
the copolymer is a block copolymer including (poly)ethylene oxide (PEO) and (poly)propylene oxide (PPO), and
implanting the piece comprises implanting the piece of the PPRh that includes the copolymer that is the block copolymer including PEO and PPO.

13. The method according to claim 12, wherein:
the block copolymer is a poloxamer, and
implanting the piece comprises implanting the piece of the PPRh that includes the block copolymer that is the poloxamer.

14. (canceled)

15. The method according to claim 1, wherein the PPRh has an elastic modulus of 0.5-5 kPa, and implanting the piece of the PPRh comprises implanting the piece of the PPRh that has the elastic modulus of 0.5-5 kPa.

16-18. (canceled)

19. The method according to claim 1, wherein:
the PPRh further includes L-lysine, and
implanting the piece of the PPRh comprises implanting the piece of the PPRh that further includes L-lysine.

20. The method according to claim 19, wherein:
the PPRh includes a solid network and a liquid component,
the L-lysine is part of the solid network, and
implanting the piece of the PPRh comprises implanting the piece of the PPRh in which the L-lysine is part of the solid network.

21. The method according to claim 19, wherein:
the PPRh includes a solid network and a liquid component,
the L-lysine is part of the liquid component, and
implanting the piece of the PPRh comprises implanting the piece of the PPRh in which the L-lysine is part of the liquid component.

22. The method according to claim 1, wherein:
the piece contains mesenchymal stem cells (MSC) suspended in the PPRh, and
implanting the piece comprises implanting the piece that contains the MSC suspended in the PPRh.

23. (canceled)

24. The method according to claim 1, wherein:
the condition includes astrogliosis, and
identifying the subject comprises identifying the subject as having the condition that includes astrogliosis.

25-27. (canceled)

28. The method according to claim 1, wherein:
the condition is a neurodegenerative disorder, and
identifying the subject as having the condition comprises identifying the subject as having the neurodegenerative disorder.

29-34. (canceled)

35. The method according to claim 1, wherein:
the condition is a traumatic CNS injury, and
identifying the subject as having the condition comprises identifying the subject as having the traumatic CNS injury.

36-39. (canceled)

40. The method according to claim 35, further comprising identifying when a traumatic incident that caused the traumatic CNS injury occurred, and wherein implanting the piece comprises implanting the piece 0-4 days after the traumatic incident occurred.

41-88. (canceled)

89. The method according to claim 15, wherein the PPRh has an elastic modulus of 0.5-1.5 kPa, and implanting the piece of the PPRh comprises implanting the piece of the PPRh that has the elastic modulus of 0.5-1.5 kPa.

90. The method according to claim 89, wherein the PPRh has an elastic modulus of about 1 kPa, and implanting the piece of the PPRh comprises implanting the piece of the PPRh that has the elastic modulus of about 1 kPa.

91. The method according to claim 15, wherein the PPRh has an elastic modulus of 2-4 kPa, and implanting the piece of the PPRh comprises implanting the piece of the PPRh that has the elastic modulus of 2-4 kPa.

92. The method according to claim 91, wherein the PPRh has an elastic modulus of about 3 kPa, and implanting the piece of the PPRh comprises implanting the piece of the PPRh that has the elastic modulus of about 3 kPa.
